(19) United States
(12) Patent Application Publication
(10) Pub. No.: US 2024/0342342 A1
(43) Pub. Date: Oct. 17, 2024

(54) NERVE GRAFTS FOR PERIPHERAL NERVE REGENERATION

(71) Applicant: VERIGRAFT AB, Goteborg (SE)

(72) Inventors: Sandra Holmgren, Halmstad (SE); Tobias Hedberg, Goteborg (SE); Lachmi Jenndahl, Goteborg (SE); Raimund Strehl, Goteborg (SE)

(73) Assignee: VERIGRAFT AB, Goteborg (SE)

(21) Appl. No.: 18/610,699

(22) Filed: Mar. 20, 2024

Related U.S. Application Data
(60) Provisional application No. 63/453,630, filed on Mar. 21, 2023.

(51) Int. Cl. A61L 27/36; A01N 1/02; A61L 101/36

(52) U.S. Cl. CPC A61L 27/3687; A01N 1/0284; A61L 27/3604; A61L 27/3675; A61L 27/3691; A61L 2101/36; A61L 2430/32; A61L 2430/40

(57) ABSTRACT

The present disclosure generally relates to nerve grafts (e.g., allografts), particularly to methods of preparing immunologically anonymized nerve segments and methods of preparing personalized nerve grafts. The present disclosure also provides methods of regenerating a nerve defect.

### CROSS-REFERENCE TO RELATED APPLICATIONS

The present application claims priority to U.S. Provisional Application No. 63/453,630, filed Mar. 21, 2023.

### BACKGROUND

The present application relates generally to the field of nerve grafts (e.g., allografts), particularly to methods of preparing immunologically anonymized nerve segments and methods of preparing personalized nerve grafts. The present disclosure also provides methods of regenerating a nerve defect.

Use of nerve allografts (e.g., a nerve from a donor subject of the same species as the subject in need thereof, but not genetically identical) to date has been discouraging, at least in part, due to immune rejection directed against, for example, Schwann cells and myelin sheaths of the graft, which can impede axonal regeneration. Thus, there is a need for improved nerve grafts (e.g., allografts) that do not illicit or induce immune rejection, and/or improve nerve defect injuries more efficiency and/or over longer distances than an appropriate reference standard (e.g., currently available nerve grafts in the art).

### SUMMARY

The present disclosure provides, among other things, methods for preparing immunologically anonymized nerve segments, methods of preparing personalized nerve grafts, and methods of regenerating a nerve defect.

In one aspect, the present disclosure provides a method of preparing an immunologically anonymized nerve segment, which may involve: non-chemically removing cells from a nerve segment; and enzymatically removing immunogenic remnants from the nerve segment to provide an immunologically anonymized nerve segment. This process is also referred to as decellularization.

In some embodiments, the nerve segment may be derived from an animal species, such as a warm-bloodied animal species, e.g. a mammal species, such as a human species.

In some embodiments, the method further may comprise prior to said non-chemically removing, snap-freezing the nerve segment. In some embodiments, snap-freezing may comprise, for example, exposing the nerve segment to a temperature below 0° C., such as a temperature below −10° C., −20° C., −30° C., −40° C., −50° C., −70° C., −90° C., −100° C., −120° C., −140° C., −160° C. or −180° C. In some embodiments, snap-freezing may involve submerging the nerve segment in a cryogenic liquid, such as liquid nitrogen.

In some embodiments, said non-chemically removing may comprise exposing the nerve segment to a salt solution having a cell disrupting effective concentration of a salt. In some embodiments, the salt may comprise KCl, MgCl₂, NaCl or a combination thereof. In some embodiments, the salt may comprise NaCl. In some embodiments, the solution may comprise the salt in a hypertonic concentration. In some embodiments, the concentration of the salt in the salt solution may be from 0.5 M to 2.0 M. In some embodiments, the exposing to the salt solution may be performed for at least 30 minutes, at least1 hour, at least 2 hours, at least 3 hours, at least 4 hours, at least 5 hours, at least 6 hours, at least 7 hours, at least 8 hours, at least 9 hours, at least 10 hours, at least 11 hours or at least 12 hours.

In some embodiments, said non-chemically removing further may comprise exposing the nerve segment to a non-osmotic liquid. In some embodiments, the non-osmotic liquid may be deionized water.

In some embodiments, the enzymatically removing may comprise exposing the nerve segment to an enzyme solution. In some embodiments, the enzyme solution comprises DNase. In some embodiments, the exposing to the enzyme solution may be performed for at least 4 hours, at least 6 hours, at least 8 hours, at least 10 hours, at least 12 hours, at least 14 hours, at least 16 hours, at least 18 hours, at least 20 hours, at least 22 hours or at least 24 hours.

In some embodiments, said non-chemically removing and said enzymatically removing are performed sequentially. In some embodiments, said non-chemically removing and said enzymatically removing are performed sequentially multiple times, such as at least two times each or at least three times each.

In some embodiments, the method may further involve eluting waste products of said non-chemically removing and/or said enzymatically removing from the nerve segment. In some embodiments, the eluting may involve washing the segment with phosphate-buffered saline. In some embodiments, said washing may be performed for at least 6 hours, at least 8 hours, at least 12 hours, at least 16 hours, at least 20 hours or at least 24 hours.

In some embodiments, the method may also involve sterilizing the immunologically anonymized nerve segment in a peracetic acid solution. In some embodiments, such sterilizing may be performed for at least 0.5 hours.

In some embodiments, the method may further involve washing the immunologically anonymized nerve segment in a buffered solution, such as a phosphate buffered solution, e.g. phosphate-buffered, saline after sterilization.

In some embodiments, the method may further involve cryopreserving the immunologically anonymized nerve graft.

In some embodiments, the present disclosure provides an immunologically anonymized nerve graft prepared according to methods described herein.

In some embodiments, the present disclosure provides a method of regenerating a nerve defect. Such method may involve implanting the immunologically anonymized nerve graft prepared by a method described herein. In some embodiments, the nerve defect may be a peripheral nerve defect. In some embodiments, the defect may be at least 1 cm long, at least 2 cm long, at least 3 cm long, at least 4 cm long, at least 5 cm long, at least 6 cm long or at least 7 cm long. In extreme cases the defect may be up to 30 cm long and require several grafts used in series.

In one aspect, the present disclosure provides a method of preparing a personalized nerve graft. The method may involve contacting a nerve graft scaffold with a suspension comprising a whole blood sample from a subject in need of the personalized nerve graft, wherein the whole blood sample is diluted in a physiological solution.

In some embodiments, certain components of the whole blood sample are selected prior to the diluting.

In one aspect, the present disclosure provides a method of preparing a personalized nerve graft. The method may involve contacting a surface of a nerve graft scaffold with an undiluted whole blood sample from a subject in need of the personalized nerve graft.

In some embodiments, the contacting is performed for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, or at least 16 days. For example, said contacting may be performed from 1 day to 16 days or from 2 days to 16 days or from 3 days to 16 days or from 3 days to 14 days or from 3 days to 12 days or from 3 days to 9 days.

In some embodiments, the nerve graft scaffold may be an immunologically anonymized nerve graft prepared according to a method described herein.

In some embodiments, the nerve graft scaffold may be an artificial nerve graft. For example, in some embodiments, the artificial nerve graft may be a 3D-printed nerve graft.

In some embodiments, a population of cells from the whole blood sample may populate the nerve graft scaffold.

In some embodiments, the whole blood sample may comprise one or more non-cellular factors. In some embodiments, uch one or more non-cellular factors of the whole blood may populate the scaffold. In some embodiments, the non-cellular factors may promote cellularization of the nerve graft scaffold and host compatibility of the nerve graft upon grafting.

In some embodiments, the suspension comprising the whole blood sample may also include an anti-thrombotic factor.

In some embodiments, the whole blood sample may be an undiluted whole blood sample. Yet in some embodiments, the whole blood sample may be a diluted whole blood sample, i.e. a whole blood sample diluted in a physiological solution. The diluted blood sample may be a suspension including the whole blood.

In some embodiments, the undiluted whole blood sample may comprise an anti-thrombotic factor.

In some embodiments, the anti-thrombotic factor may include an anticoagulant agent. In some embodiments, the anticoagulant agent may include a heparin or a dextran. In some embodiments, a concentration of the heparin in the suspension comprising the whole blood sample or in the undiluted whole blood sample may be from about 0.1 IU/mL to about 200 IU to mL or from about 0.5 IU/mL to about 150 IU/mL, or from about 1 IU/mL to about 140 IU/mL or from about 10 IU/mL to about 120 IU/mL or from about 30 IU/mL to about 100 IU/mL or from about 40 IU/mL to about 70 IU/mL or about 50 IU/mL at the beginning of contacting the surface of the nerve graft scaffold.

In some embodiments, the anti-thrombotic factor comprises acetylsalicylic acid. In some embodiments, a concentration of the acetylsalicylic acid in the suspension comprising the whole blood sample or in the undiluted whole blood sample is from about 0.2 μg/mL to about 200 μg/mL or from about 0.5 μg/mL to about 150 μg/mL or from about 1 μg/mL to about 100 μg/mL or from about 2 μg/mL to about 50 μg/mL or from about 3 μg/mL to about 10 μg/mL or about 5 μg/mL at the beginning of contacting the surface of the nerve graft scaffold.

In some embodiments, the suspension comprising whole blood components or the undiluted whole blood sample further may include equal to or more than the population average physiological level of one or more growth factors selected from the group consisting of: a granulocyte macrophage-colony stimulating factor (GM-CSF), interleukin (IL)-3, IL-4, neutrophin (NT)-6, pleiotrophin (HB-GAM), midkine (MK), interferon inducible protein-10 (IP-10), platelet factor (PF)-4, monocyte chemotactic protein-1 (MCP-1), RANTES (CCL-5, chemokine (C-C motif) ligand 5), IL-8, IGFs, a fibroblast growth factor (FGF)-1, FGF-2, FGF-3, FGF-4, FGF-5, FGF-6, FGF-7, FGF-8, FGF-9, a transforming growth factor (TGF)-β, a vascular endothelial growth factor (VEGF), platelet-derived growth factor (PDGF)-A, PDGF-B, HB-EGF, hepatocyte growth factor (HGF), tumor necrosis factor (TNF)-α, insulin-like growth factor (IGF)-1, and any combination(s) thereof.

In some embodiments, the one or more growth factors may include a fibroblast growth factor-2. In some embodiments, the one or more factors comprises a vascular endothelial growth factor. In some embodiments, the one or more growth factors comprise a fibroblast growth factor-2 and a vascular endothelial growth factor.

In some embodiments, the fibroblast growth factor is rhFGF-2. In some embodiments, a concentration of the rhFGF-2 in the undiluted whole blood sample may be from about 1 ng/mL to about 100 ng/mL or from about 2 ng/mL to about 70 ng/ml or from about 3 ng/ml to about 50 ng/ml or from about 5 ng/ml to about 30 ng/mL at the beginning of contacting the surface of the nerve graft scaffold.

In some embodiments, the vascular endothelial growth factor is rhVEGF. In some embodiments, a concentration of the rhVEGF in the undiluted whole blood sample may be from about 1 ng/mL to about 150 ng/ml or from about 5 ng/ml to about 120 ng/ml or from about 10 ng/ml to about 100 ng/mL or from about 20 ng/mL to about 100 ng/ml or from about 30 ng/ml to about 100 ng/ml at the beginning of contacting the surface of the nerve graft scaffold.

In some embodiments, the contacting may be performed in a vessel. In some embodiments, the method further involves coating the vessel containing the nerve graft scaffold with an anticoagulant prior to initiating the contacting. In some embodiments, the anticoagulant coating may include heparin.

In some embodiments, said contacting is performed for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days up to 14 days.

In some embodiments, the method may also involve measuring a concentration of D-glucose in the suspension comprising the whole blood every day of the contacting and adding D-glucose to the suspension to maintain the concentration of D-glucose in the suspension at a certain level, such as at least 1 mmol/L or at least 2 mmol/L or at least 3 mmol/L or at least 4 mmol/L or at least 5 mmol/L. In some embodiments, the maintained concentration of D-glucose in the suspension may be the between 3 mmol/L and 11 mmol/L.

In some embodiments, a method of regenerating a nerve defect may involve implanting the personalized nerve graft prepared by a method described herein to the subject, whose blood was used for preparing the personalized nerve graft. In some embodiments, the nerve defect is a peripheral nerve defect. In some embodiments, the defect may be at least 1 cm long, at least 2 cm long, at least 3 cm long, at least 4 cm long, at least 5 cm long, at least 6 cm long or at least 7 cm long. In extreme cases the defect may be up to 30 cm long and require several grafts used in series.

In some embodiments, a method described herein is repeated (e.g., conducted more than one time, including, for example, 2 times, 3 times, 4 times, 5 times, 6 times).

### FIGURES

FIG. 1A-1D are photographs showing an immunologically anonymized nerve allograft. FIG. 1A is a photograph that shows a nerve allograft that was trimmed into 7-cm long nerve segment. FIG. 1B is a photograph that shows the trimmed nerve allograft being placed into the gap created in the common peroneal nerve. FIG. 1C-1D are photographs that show that the immunologically anonymized nerve allograft (FIG. 1C) or the same nerve segment resected used as autograft (FIG. 1D) were sutured bridging the gap.

FIG. 2A-2J are representative micrograph images showing immunofluorescent staining of myelinated axons (FIG. 2A, 2F), Schwann cells (FIG. 2B, 2G), nuclei (FIG. 2C, 2H), extracellular matrix proteins (FIG. 2D, 2I) and myelin (FIG. 2E, 2J), in a control nerve (FIG. 2A, 2B, 2C, 2D, 2E) and in a decellularized sheep nerve graft (FIG. 2F, 2G, 2H, 2I, 2J). The images were taken at 200× magnification; scale bar 150 μm.

FIG. 3A-3F relate to evaluation of nerve regeneration. FIG. 3A-C present plots related to electrophysiological evaluation of nerve regeneration along 120 days follow-up after sciatic nerve section and repair. Results are presented as mean±SEM. Statistical analysis was performed using 2-way ANOVA. The plots show the amplitude of CAMP of plantar (*p<0.05 vS AG), tibialis anterior (*p<0.05 vs AG) and gastrocnemius (*p<0.05 vs AG) muscles. FIG. 3D shows representative micrographs showing immunofluorescent staining of myelinated axons labeled against Neurofilament 200, Schwann cells labeled against S100, nuclei labeled with DAPI, macrophages labeled with IBA1 and extracellular matrix labeled with laminin in cross sections of nerve graft in control, AG (autologous graft) and DRA (immunologically anonymous) groups at ×20 magnification; scale bar 200 μm. FIG. 3E shows representative transverse semithin sections of the mid graft and distal to the graft in AG and DRA groups, stained with toluidine blue. Images were taken at ×1000 magnification; scale bar 10 [symbol] m. FIG. 3F is a plot that shows the density of myelinated axons in the sciatic nerve at the mid graft and distal to the graft in the two groups from FIG. 3E. **p<0.01 vs AG.

FIG. 4A-D shows plots of the functional evaluation along the 9 months follow-up in the two groups of sheep. Similar evolution in the groups AG (autologous graft) and DC (immunologically anonymous) was observed for all the tests applied.

FIG. 5A-5P demonstrates evaluation of a control sheep and a grafted sheep. FIG. 5A-5C shows macroscopic representative pictures of a control sheep peroneal nerve (FIG. 5A), a peroneal nerve autograft (FIG. 5B), and an immunologically anonymized nerve allograft (FIG. 5C), both with a neuroma visible at proximal and distal sutures, harvested at the end of the study. FIG. 5D shows schema of the division in sections of nerves harvested. Section 1 includes the proximal suture and section 3 includes the distal suture of the graft. Micrographs of cross sections of the proximal segment of the graft of sheep stained with hematoxylin and eosin. FIG. 5E and 5H show a control nerve, FIG. 5F and 5I show an autograft and FIG. 5G and 5J show an immunologically anonymized nerve graft, at ×40 magnification (FIG. 5E, 5F, 5G), scale bar 200 μm, and at ×200 magnification (FIG. 5H, 5I, 5J), scale bar 100 μm. Representative semithin transverse sections of the graft of sheep in AG and DC groups, stained with toluidine blue. FIG. 5K and 5N show control nerve, FIG. 5L and 5O show an autograft and FIG. 5M and 5P show an immunologically anonymized nerve graft, at 40× magnification (FIG. 5K, 5L, 5M), scale bar 200 [symbol] m and at ×400 magnification (FIG. 5N, 5O, 5P), scale bar 100 [symbol] m.

FIG. 6A-6L shows representative micrographs showing immunofluorescence of myelinated axons labeled against Neurofilament 200 in cross sections of the proximal graft in AG and DC groups. FIG. 6A and 6D show control nerve, FIG. 6B and 6E show an autograft and FIG. 6C and 6F show an immunologically anonymized nerve graft, at ×40 magnification (FIG. 6A, 6B, 6C), scale bar 200 μm, and at ×400 magnification (FIG. 6D, 6E, 6F), scale bar 100 μm. Representative micrographs showing immunofluorescence labeling of Schwann cells with an antibody against the S100 protein in cross sections of the proximal graft in AG and DC groups. FIG. 6G and 6J show control nerve, FIG. 6H and 6K show autograft and FIGS. 6I and 6L show an immunologically anonymized nerve graft at ×100 magnification (FIG. 6G, 6H, 6I), scale bar 100 μm, and at ×400 magnification (FIG. 6J, 6K, 6L), scale bar 100 μm.

FIG. 7A-7L shows representative micrographs. Representative micrographs of cross sections of the Tibialis Anterior muscle stained with hematoxylin and eosin from (FIG. 7A, 7D) the contralateral side (control), (FIG. 7B, 7E) an animal from AG group, (FIG. 7C, 7F) an animal from DC group, at ×40 magnification (FIG. 7A, 7B, 7C), scale bar 500 μm, and at ×200 magnification (FIG. 7E, 7F, 7G), scale bar 150 μm. Representative micrographs of perpendicular sections of the skin of the dorsum of the foot stained with hematoxylin and eosin from (FIG. 7G, 7J) the contralateral side (control), (FIG. 7H, 7K) an animal from AG group, (FIG. 7I, 7L) an animal from DC group, taken at ×40 magnification (FIG. 7G, 7H, 7I), scale bar 200 μm, and at ×200 magnification (FIG. 7J, 7K, 7L), scale bar 100 μm.

FIG. 8A-8B show images of the proprioception test and the flexor withdrawal reflex. The proprioception test measures the capability of dorsiflexion of the hindfoot from a plantarflexion position (FIG. 8A). The flexor withdrawal reflex measures the sensory response to pinching the dorsum of the foot in a proximal point, middle point (FIG. 8B) or distal point.

FIG. 9A-9F show representative EMG recordings and ecographic images. Representative EMG recordings of the CMAP recorded in the TA muscle evoked by stimulation of the sciatic nerve in the intact left hindlimb (FIG. 9A) and in the operated right hindlimb of a sheep at 6.5 (FIG. 9B) and 9 (FIG. 9C) months after operation. Label “2” is at the peak of the CMAP. Scale in A: amplitude 10 mV/square and time 3 ms per square; in B and C: amplitude 500 μV/square and time 5 ms per square. Representative ecographic images of the TA muscle recorded in the intact left hindlimb (FIG. 9D) and in the operated right hindlimb of a sheep repaired with an autograft (FIG. 9E) or with an immunologically anonymized nerve allograft (FIG. 9F).

FIG. 10A-10D show representative micrographs of cross sections of nerve segments at 10× magnification. Hematoxylin/Eosin (A, B) and 40,6-diamidino-2-phenylindole (DAPI) staining (C, D) of explanted nerve segment (A, C), and immunologically anonymized nerve segment (B, D). Nuclei were visible in the native nerve segment whereas no or few nuclei were visible after processing.

FIG. 11 is a plot showing DNA content quantified in native explanted nerve segments (Native) and immunologically anonymized nerve grafts (immunologically anonymized) described as average +/− standard error of the mean (n=7). DNA quantification revealed almost complete removal of DNA during the anonymization process (0.58±0.08 ng/mg tissue) compared with native explanted nerve segments (195±44 ng/mg tissue). Remaining DNA represents only around 0.3% of the native DNA content.

FIG. 12A-12F are representative micrographs of cross sections of nerve segments at 10× magnification. Hematoxylin/Eosin (A-C) and 40,6-diamidino-2-phenylindole (DAPI) staining (D-F) of native explanted nerve segment (A, D), immunologically anonymized nerve graft (B; E) and personalized nerve graft (C, F). Cell nuclei were visible in the native explanted nerve segment as well as in the personalized nerve graft whereas no or few nuclei were visible in the immunologically anonymized nerve graft.

FIG. 13 is a plot showing DNA content quantified in native explanted nerve segment (Native), immunologically anonymized nerve graft (immunologically anonymized) and personalized nerve graft (Personalized), described as average +/− standard error of the mean (n=4). DNA quantification revealed almost complete removal of DNA in the immunologically anonymized nerve graft (0.46±0.05 ng/mg tissue) compared with the native explanted nerve segment (107±6.6 ng/mg tissue). In the personalized nerve graft, DNA content in the graft was increased to 29±7.4 ng/mg tissue (FIG. 13).

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

In practicing the present technologies, many conventional techniques in molecular biology, protein biochemistry, cell biology, microbiology and recombinant DNA are used. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins 10 eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Nerve defects (e.g., nerve injuries, such as peripheral nerve injuries) can result in partial or total loss of the sensory, autonomic and/or motor functions dependent on the injured nerve, with important consequences for the quality of life of affected patients (Navarro et al., Progress in Neurobiology 82 (4), 163-201 (2007)). It is estimated that more than 300,000 persons are affected by traumatic nerve injury every year in North America and Europe (Noble et al., Journal of Trauma, 45 (1), 116-122 (1998); Robinson, Muscle & Nerve, 23 (6), 863-873 (2000)). Although peripheral neurons are able to regenerate after axotomy (e.g., cutting or otherwise severing of an axon), and eventually reinnervate target organs, functional recovery is often unsatisfactory, especially following severe injuries (Allodi et al., Journal of Neuroscience Methods 198 (1), 53-61 (2011); Pfister et al., Critical Reviews in Biomedical Engineering, 39 (2), 81-124, (2011)). It is understood that poor functional outcomes generally stem from, at least in part, long regenerative distances coupled with relatively slow rate of axonal regeneration (˜1-2 mm/day), creating prolonged periods of denervation that ultimately limit the regenerative capacity of the distal nerve structure (Burrell et al., Surgical approach, recovery kinetics, and clinical relevance. Neurosurgery 87 (4), 833-846 (2020); Gordon et al., Journal of Neuroscience 31 (14), 5325-5334 (2011)). Therefore, challenges regarding recovery after nerve defect (e.g., peripheral nerve injury) include long distance nerve defects and proximal injuries in long limbs.

When the length of the gap created by a nerve defect is too long to allow apposition and direct suture without tension, nerve grafts are usually employed for repairing. Without wishing to be bound by any one theory, the nerve graft between the stumps of a transected (e.g., severed) nerve is understood to offer mechanical guidance, and the Schwann cells of the graft and their basal lamina can play an important role in promoting axonal growth (Hall, Neuropathology and Applied Neurobiology, 12 (1), 27-46 (1986)). Currently, autologous nerve grafts (also referred to as “autografts”) are understood to be the “gold standard” surgical treatment for complex peripheral nerve injuries in the clinic; however, autografts can present a number of limitations. Apart from the paucity of expendable nerve tissue, harvesting autologous nerves can result in significant donor site morbidity, increased risk of infection, and/or longer intraoperative times (Moore et al., Muscle and Nerve 44 (2), 221-234 (2011)). The alternative use of allografts has been discouraging to date, at least in part due to the immune rejection directed against Schwann cells and myelin sheaths of the graft which is understood to impede axonal regeneration (Evans et al., Progress in Neurobiology, 43 (3), 187-233 (1994)). Processed acellular nerve allograft have been promoted as a potential replacement (Isaacs and Safa, Hand 12 (1), 55-59 (2017); Moore et al., Muscle and Nerve 44 (2), 221-234 (2011)). If adequately decellularized, it is understood that such grafts do not induce immune response, whereas the internal nerve structure, including endoneurial tubules, basal lamina and extracellular matrix (ECM) components, remains supporting axonal regeneration, despite the absence of the activated Schwann cells (Hall, Neuropathology and Applied Neurobiology, 12 (1), 27-46 (1986)).

The present disclosure is based on, among other things, improved methods of preparing allografts, including, for example, immunologically anonymized nerve segments and personalized nerve grafts, as described herein. The present disclosure also provides methods of regenerating a nerve defect. Technologies of the present disclosure provide, among other things, a solution to the limited ability of nerve grafts (e.g., allografts) to successfully repair and/or regenerate nerve defects (e.g., of a certain length, to a certain degree of restored functionality) relative to an appropriate reference standard (e.g., known methods of nerve grafting, such as autografts).

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

Unless otherwise specified, “a” or “an” means one or more.

As used herein, the term “about” placed before a specific numeric value may mean ±20% of the numeric value; ±18% of the numeric value, ±15% of the numeric value; ±12% of the numeric value; ±8% of the numeric value; ±5% of the numeric value; ±3% of the numeric value; ±2% of the numeric value; ±1% of the numeric value or ±0.5% of the numeric value.

As used herein, the term “administering” of an agent to a subject includes any route of introducing or delivering the agent to the subject to perform its intended function. Administration can be carried out by any suitable route, including, but not limited to, intravenously, intramuscularly, intraperitoneally, subcutaneously, and other suitable routes as described herein. Administration includes self-administration and the administration by another.

As used herein, the term “allograft”, in the context of nerve grafts of the present disclosure, means that the nerve graft is prepared with a section of nerve tissue (e.g., a nerve segment) from one subject (e.g., a donor subject) of the same species as the subject in need thereof who is not genetically identical. As used herein, the term “xenograft”, in the context of nerve grafts of the present disclosure, means that the nerve graft is prepared with a section of nerve tissue (e.g., a nerve segment) from one subject (e.g., a donor subject) of a different species as the subject in need thereof who is not genetically identical. This is distinct from “autograft”, where the subject (e.g., donor subject) of the section of nerve tissue and the subject in need thereof are genetically identical (e.g., are the same subject).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “donor subject” refers to the source of an allograft oe xenograft. A donor subject can be any suitable animal, including, for example, humans, cows, sheep, mouse, rat, non-human primates, horse, dog, cat, etc. A donor subject can also be a cadaver.

As used herein, the term “acellular” or “decellularized” refers to a tissue (e.g., nerve graft) in which cells are removed and/or disintegrated. The level of cell removal and/or disintegration can depend on the exact source of tissue, the methodology used to extract the cell, and the need for the removal and/or disintegration of cells. The amount of cell removal and/or disintegration (e.g., decellularization) can range from removal and/or disintegration of a couple percent of cells (e.g., 2%) to removal nearly 100 percent or 100 percent. The resulting tissue scaffold is referred to as an immunologically anonymized nerve segment.

As used herein, the term “nerve graft” refers to a piece of nerve tissue that serves as a bridge to fill a gap between two ends of a nerve defect (e.g., a damaged nerve, a severed nerve). A “nerve graft” can be an “autograft.” A “nerve graft” can be an “allograft.” A nerve graft can be derived from any suitable animal source, including, for example, humans, cows, sheep, mouse, rat, non-human primates, horse, dog, cat, etc.

As used herein, the term “nerve graft scaffold” refers to a structure, such as an immunologically anonymized nerve segment, e.g. a decellularized nerve segment, that can guide and/or transmit axons to a distal location and/or minimize the probability of aberrant neural sprouting.

As used herein, the term “reduce” or “decrease” means to alter negatively by at least about 5% including, but not limited to, alter negatively by about 5%, by about 10%, by about 25%, by about 30%, by about 50%, by about 75%, or by about 100%.

### Immunologically Anonymized Nerve Segments

In one aspect, the present disclosure provides, among other things, immunologically anonymized nerve segments and methods of preparing and/or characterizing immunologically anonymized nerve segments. An immunologically anonymized nerve segment can be a nerve segment comprising the absence or near absence of cells (e.g., decellularized), such as, for example and without limitation, Schwann cells, fibroblasts, and pericytes. Extracellular matrix (ECM) components (e.g., constituting the nerve fiber) can be substantially retained in the immunologically anonymized nerve segment compared to an appropriate reference standard (e.g., ECM components of a nerve segment that has not been decellularized).

### Methods of Preparing an Immunologically Anonymized Nerve Segment

The present disclosure provides, among other things, a plurality of methods of preparing an immunologically anonymized nerve segment. An immunologically anonymized nerve segment can be prepared by decellularizing a nerve segment and/or removing immunogenic components from a donor subject, while substantially retaining the ECM components.

A donor subject can be any suitable species. In some embodiments, a donor subject can be an animal species, such as a warm-blooded animal species, e.g. a mammal species, such as a human species. In some embodiments, the mammal, for example, may be a human, a non-human primate (e.g., orangutan, baboon, or chimpanzees), horses, cattle, pigs, sheep, goats, llama, dogs, cats, rabbits, guinea pigs, gerbils, hamsters, rats, and mice. In some embodiments, a donor subject may be a cadaver. In some embodiments, the nerve segment may be derived (e.g., surgically removed) from an animal species, such as a warm-blooded animal species, e.g., a mammal species, such as a human species.

A method of preparing an immunologically anonymized nerve segment can comprise: non-chemically removing cells from a nerve segment (e.g., decellularizing a nerve segment, such as a nerve segment sourced from a donor subject); and enzymatically removing immunogenic remnants from the nerve segment to provide an immunologically anonymized nerve segment. In some embodiments, non-chemically removing cells from a nerve segment and enzymatically removing immunogenic remnants from the nerve segment (e.g., to provide an immunologically anonymized nerve segment) can be performed sequentially. In some embodiments, non-chemically removing cells from a nerve segment and enzymatically removing immunogenic remnants from the nerve segment (e.g., to provide an immunologically anonymized nerve segment) can be performed sequentially at least two times each (including, for example, 2 times, 3 times, 4 times, 5 times, or 6 times).

The method of preparing an immunologically anonymized nerve segment can further comprise, prior to non-chemically removing cells from a nerve segment, snap-freezing the nerve segment. In some embodiments, snap-freezing comprises submerging the nerve segment in a cryogenic liquid, such as liquid nitrogen, and optionally storing the nerve a temperature below 0° C., such as a temperature below −10° C., −20° C., −30° C., −40° C., −50° C., −70° C., −80° C., −90° C., −100° C., −120° C., −140° C., −160° C. or-180° C. (e.g., until decellularizing the nerve segment).

Non-chemically removing cells from a nerve segment (e.g., decellularizing a nerve segment, such as a nerve segment sourced from a donor subject) can comprise exposing a nerve segment to a high osmolarity solution, such as a salt solution having a cell disrupting effective concentration of a salt. In some embodiments, a salt in the salt solution may be or may include KCl, MgCl₂, NaCl, or a combination thereof. In some embodiments, a salt in the salt solution may be or may include NaCl. In some embodiments, a salt in the salt solution may be or may include KCl. In some embodiments, a salt in the salt may be or may include MgCl₂. In some embodiments, the concentration of the salt in the salt solution may be about 0.5 M, about 0.6 M, about 0.7 M, about 0.8 M, about 0.9 M, about 1.0 M, about 1.1 M, about 1.2 M, about 1.3 M, about 1.4 M, about 1.5 M, about 1.6 M, about 1.7 M, about 1.8 M, about 1.9 M, or about 2.0 M. In some embodiments, the concentration of the salt in the salt solution may be about 0.5 M to about 2.0 M, about 0.8 M to about 2.0 M, about 1.0 M to about 2.0 M, about 1.2 M to about 2.0 M, about 1.5 M to about 2.0 M, about 0.5 M to about 1.5 M, about 0.8 M to about 1.5 M, or about 1.0 M to about 1.5 M. In some embodiments, the concentration of the salt in the salt solution is about 1.0 M.

In some embodiments, exposing a nerve segment to a salt solution may be performed for at least 30 minutes, at least 1 hour, at least 3 hours, at least 6 hours, at least 12 hours, at least 18 hours, or at least 24 hours. In some embodiments, exposing a nerve segment to a salt solution may be performed for about 30 minutes to about 24 hours, about 1 hour to about 24 hours, about 3 hours to about 24 hours, about 6 hours to about 24 hours, about 12 hours to about 24 hours, about 18 hours to about 24 hours, about 1 hour to about 18 hours, about 1 hour to about 12 hours, about 3 hours to about 18 hours, or about 3 hours to about 12 hours.

Methods of preparing an immunologically anonymized nerve segment may further involve, after non-chemically removing cells from a nerve segment, exposing the nerve segment to a non-osmotic or a low osmolarity liquid. A non-osmotic or a low osmolarity liquid can include, for example and without limitation, water (e.g., deionized water, ultrapure water), phosphate buffered saline (PBS), or any combination thereof. In some embodiments, exposing the nerve segment to a non-osmotic or low osmolarity liquid is performed for a certain period of time. In some embodiments, the certain period of time may be about 1 minute, about 2 minutes, about 5 minutes, about 10 minutes, about 15 minutes, about 30 minutes, about 45 minutes, about 60 minutes, about 75 minutes, about 90 minutes, about 105 minutes, or about 120 minutes. In some embodiments, exposing the nerve segment to a non-osmotic or low osmolarity liquid may be performed for about 5 minutes. In some embodiments, exposing the nerve segment to a non-osmotic or low osmolarity liquid may be performed for about 120 minutes. The nerve segment may be exposed to a non-osmotic or a low osmolarity liquid a plurality of times for the certain period of time. In some embodiments, the nerve segment is exposed to a non-osmotic or a low osmolarity liquid one time, two times, three times, four times, or five times.

Enzymatically removing immunogenic remnants (e.g., DNA) from the nerve segment (e.g., to provide an immunologically anonymized nerve segment) may comprise exposing the nerve segment to an enzyme solution. The enzyme solution may comprise one or more enzymes such as deoxyribonuclease (DNase), Dispase and Pepsin. In some embodiments, the enzyme solution may contain DNase I. In some embodiments, the enzyme solution may contain DNase II. In some embodiments, the enzyme solution may contain DNase I and DNase II. The enzyme solution may also contain a buffer solution, such as a saline buffer solution, such as phosphate-buffered saline (PBS), which may be for example, PBS containing calcium chloride and magnesium chloride. In some embodiments, the enzyme solution comprises about 20 U/mL DNase, about 30 U/mL DNase, about 40 U/mL DNase, about 50 U/mL DNase, about 60 U/mL DNase, about 70 U/mL DNase, or about 80 U/mL DNase. In some embodiments, the enzyme solution comprises about 30 U/mL DNase. In some embodiments, the enzyme solution comprises about 40 U/mL DNase. In some embodiments, the enzyme solution comprises about 50 U/mL DNase. In some embodiments, the enzyme solution comprises about 20 U/mL to about 80 U/mL DNase, about 20 U/mL to about 70 U/mL DNase, about 20 U/mL to about 60 U/mL DNase, about 20 U/mL to about 50 U/mL DNase, about 30 U/mL to about 80 U/mL DNase, about 30 U/mL to about 70 U/mL DNase, about 30 U/mL to about 60 U/mL DNase, or about 30 U/mL to about 50 U/mL DNase. In some embodiments, exposing the nerve segment to the enzyme solution may be performed for at least 4 hours, at least 8 hours, at least 12 ours, at least 16 hours, or at least 24 hours. In some embodiments, exposing the nerve segment to the enzyme solution may be performed for about 15 hours, about 16 hours, about 17 hours, about 18 hours, about 19 hours, or about 20 hours. In some embodiments, a certain period of time is about 15 to about 20 hours.

Enzymatically removing immunogenic remnants from the nerve segment may, in some embodiments, be followed by contacting the nerve segment with a wash solution (e.g., water, deionized water, ultrapure water, PBS). The nerve segment can be contacted with a wash solution one time, two times, three times, four times, or five times each time for about 1 minute, about 2 minutes, about 3 minutes, about 4 minutes, about 5 minutes, about 6 minutes, about 7 minutes, about 8 minutes, about 9 minutes, or about 10 minutes.

Methods of preparing an immunologically anonymized nerve segment can further comprise eluting waste products, such as Schwann cells, myelin sheath, DNA, cell membrane components, intracellular components, immunogenic proteins, glycoproteins (e.g., HLA, MHC), of non-chemically removing and/or enzymatically removing from the nerve segment. In some embodiments, eluting comprises washing the segment with a buffer solution, such as PBS. In some embodiments, such washing may be performed for at least 6 hours, at least 12 hours, or at least 24 hours. In some embodiments, the washing may be performed for about 6-12 hours, about 12-24 hours, or about 6-24 hours. In some embodiments, washing is performed for about 6 hours. In some embodiments, washing is performed for about 12 hours. In some embodiments, washing is performed for about 24 hours.

Methods of preparing an immunologically anonymized nerve segment can further comprise sterilizing the immunologically anonymized nerve segment. Sterilizing the immunologically anonymized nerve segment can include contacting the immunologically anonymized nerve segment with a peracetic acid solution. A concentration of the peracetic acid in such solution may vary. In some embodiments, the concentration of the peracetic acid may be from 0.01% to 1.0% or from 0.02% to 0.5% or from 0.03% to 0.3% or from 0.05% to 0.2% or about 0.1%. In some embodiments, sterilizing (e.g., with peracetic acid solution) may be performed for at least 0.5 hours, at least 1 hour, at least 2 hours, at least 5 hours, at least 10 hours, at least 12 hours, at least 15 hours, at least 20 hours, or at least 24 hours. In some embodiments, sterilizing can be performed for about 0.5-24 hours, about 0.5-20 hours, about 0.5-15 hours, about 0.5-10 hours, about 1-24 hours, about 1-15 hours, about 1-10 hours, about 5-24 hours, about 5-20 hours, or about 5-15 hours. Sterilizing the immunologically anonymized nerve segment can further comprise washing the immunologically anonymized nerve segment in phosphate-buffered saline after sterilization. A plurality of washes can be completed over the course of sterilization (e.g., 1 wash, 2 washes, 3 washes, 4 washes, 5 washes).

Methods of preparing an immunologically anonymized nerve segments can further comprise cryopreserving the immunologically anonymized nerve graft (e.g., by snap-freezing in liquid nitrogen) and optionally storing the cryopreserved immunologically anonymized nerve graft for a certain period of time, such as for at least 1 day, at least 1 week, at least 2 weeks, at least 1 month, at least 3 months, at least 6 months, at least 9 months, at least 12 months, at least 18 months, at least two years, at least three years or any period within these ranges.

Following non-chemically removing cells from a nerve segment and enzymatically removing immunogenic remnants from the nerve segment, the nerve segment may be characterized (e.g., quality controlled) to confirm an immunologically anonymized nerve segment (e.g., a decellularized nerve segment comprising substantially retained ECM components) was produced by the methods described herein. If a nerve segment is characterized as not an immunologically anonymized nerve segment, the process(es) may be repeated on the same nerve segment or the nerve segment may be discarded and the process(es) described herein repeated with a new native nerve segment (e.g., a nerve segment which has not undergone methods described herein). If a nerve segment is characterized as an immunologically anonymized nerve segment, the immunologically anonymized nerve segment can be further utilized or undergo other methods (e.g., personalization) as described herein.

In another aspect, the present disclosure provides an immunologically anonymized nerve graft prepared according to the methods described herein.

An immunologically anonymized nerve segment can also be prepared by assembling a biocompatible material in vitro, for example, using bioprinting or polymer self-assembly. (e.g., an artificial nerve graft). In some embodiments, the artificial nerve graft is a 3D-printed nerve graft. A plurality of methods of assembling a biocompatible material in vitro (e.g., bioprinting or polymer self-assembly) are known and understood in the art and one of ordinary skill in the art could readily select and use such methods in the art in accordance with technologies of the present disclosure.

### Methods of Characterizing an Immunologically Anonymized Nerve Segment

A plurality of methods may be utilized to characterize an immunologically anonymized nerve segment. A nerve segment (e.g., an immunologically anonymized nerve segment) may be characterized at any one or more points during a method of preparing an immunologically anonymized nerve segments as described herein. Alternatively, or additionally, an immunologically anonymized nerve segment (e.g., prepared according to methods of the present disclosure) may be characterized after preparation, i.e. after the preparation is complete.

Characterization can include determining whether or not a nerve segment was successfully anonymized. Successful immunological anonymization of a nerve segment can be wherein the nerve segment was decellularized and/or comprised substantially retained ECM components. In some embodiments, successful anonymization of a nerve segment is determined as the absence or near absence of cells (e.g., absence of detectable cells) from the nerve segment, such as the absence or near absence of, for example, Schwann cells, fibroblasts, pericytes, and/or any other cell type (e.g., cell type that initiates an undesired immune rejection) which may be present in the nerve segment (e.g., from a donor subject). Characterizing the presence or absence or near absence of cells (e.g., absence of detectable cells) can be completed according to a plurality of methods readily known and understood in the art. For example, and without limitation, staining nuclei in histological sections using standard histological staining procedures, fluorescent staining (e.g., DAPI straining) and microscopy, and/or DNA quantification can be utilized.

Immunological anonymization processes, such as non-chemically removing cells from a nerve segment, can also remove cell membranes (e.g., comprising HLA class-I antigens and/or HLA class-II antigens). The absence of cell membranes or components thereof, such as HLA class-I antigens and/or HLA class-II antigens, can also be characterized using methods known in the art.

Extracellular matrix (ECM) components, such as nerve fiber, can be substantially retained compared to an appropriate reference standard (e.g., ECM components of a nerve segment that has not been anonymized) throughout methods of preparing an immunologically anonymized nerve segment as described herein. A nerve segment (e.g., an immunologically anonymized nerve segment) can be characterized at any one or more points during a method of preparing an immunologically anonymized nerve segments for the morphology and architecture of, for example, the epineurium and perineurium and fascicle integrity can be examined. Such features can be examined, for example and without limitation, visually and/or with histological analysis (e.g., by staining with Hematoxylin Eosin (H&E) to verify that methodologies described herein has not compromised the three-dimensional structure and/or bioactivity of the ECM scaffold). Histological analysis by staining the anonymized nerve graft can also be useful to visualize architecture and removal of cell compositions, for example, by using nuclear staining. Antibodies against S-100 protein and myelin binding protein (MBP), flouromyelin, can be used to check maintenance of neural structures. Without wishing to be bound by any one theory, the presence of an intact ECM (e.g., relative to an appropriate comparator, such as a nerve segment that did not undergo methods of preparing an immunologically anonymized nerve segment described herein) following methods of preparing an immunologically anonymized nerve segment is understood to be important to allow axonal guidance during regenerating a nerve defect (e.g., a peripheral nerve defect).

A nerve segment that has been decellularized (e.g., absence or near absence of detectable cells) and/or comprises a substantially retained ECM compared to an appropriate reference standard (e.g., ECM components of a nerve segment that has not been decellularized) is characterized as an immunologically anonymized nerve segment. When a nerve segment is characterized as an immunologically anonymized nerve segment, the immunologically anonymized nerve segment can be further processed (e.g., cryopreserved, personalized).

A nerve segment comprising one or more detectable cells above an accepted limit in the nerve segment is characterized as a non-immunologically anonymized nerve segment. When a nerve segment is characterized as a non-immunologically anonymized nerve segment can be discarded and, optionally, a new donor nerve segment can be selected to undergo methods of preparing an immunologically anonymized nerve segment as described herein.

### Personalized Nerve Grafts

Nerve graft scaffolds, such as immunologically anonymized nerve segments and artificial nerve grafts (e.g., produced in accordance with the methods described herein), can be personalized to thereby produce personalized nerve grafts. Personalization can, for example, increase immunocompatibility and/or add additional features of guidance and/or attraction in axonal regrowth of the nerve graft.

An immunologically anonymized nerve graft can be personalized with biological components, such as blood, extracellular vesicles (micelles), specific proteins, chemo attractants and/or growth factors, from a subject (e.g., a subject in need thereof) to, for example, increase biocompatibility and/or improve the process of axonal regrowth through the nerve graft post implantation.

### Methods of Preparing Personalized Nerve Grafts

The present disclosure provides, among other things, a plurality of methods of preparing personalized nerve grafts from a nerve graft scaffold (also referred to herein as “personalization”). A nerve graft scaffold can be an immunologically anonymized nerve segment or an artificial nerve graft (e.g., a 3D-printed nerve graft) as described elsewhere herein.

Personalization can be achieved, for example, utilizing processes described for personalization of blood vessels (e.g., tubular structures) as described in WO2020026212, incorporated herein by reference in its entirety.

In one aspect, the present disclosure provides methods of preparing a personalized nerve graft comprising contacting a nerve graft scaffold (e.g., an immunologically anonymized nerve segment, an artificial nerve graft) with a suspension comprising a whole blood sample from a subject in need of the personalized nerve graft, wherein the whole blood sample is diluted in a physiological solution.

The whole blood sample can be diluted with a physiological solution. In some embodiments, the physiological solution may be a solution that is iso-oncotic with blood. In some embodiments, the physiological solution may be or may contain STEEN™ solution (e.g., a solution comprising Human Serum Albumin (HSA), Dextran 40, and physiological salt solution). In some embodiments, the whole blood sample is diluted at a ratio of whole blood: physiological solution at a ratio of about 0.25:1, about 0.5:1, about 0.75:1, about 1:1, about 1:1.25, about 1:1.5, about 1:1.75, or about 1:2. In some embodiments, the whole blood sample is diluted at a ratio of whole blood: physiological solution at a ratio of about 0.25:1 to about 1:2, of about 0.5:1 to about 1:2, of about 1:1 to about 1:2, or of about 0.5:1 to about 1:1.5.

In some embodiments, platelet-inhibitors and/or anticoagulants are added to the physiological solution. Platelet-inhibitors and/or anticoagulants can include, for example, Acetylsalicylic acid (ASA), heparin, including low molecular weight heparin. In some embodiments, ASA may be added to the physiological solution at a concentration of about 3 μg/mL, about 4 μg/mL, about 5 μg/mL, about 6 μg/mL, about 7 μg/mL, about 8 μg/mL, about 9 μg/mL, or about 10 μg/mL. In some embodiments, ASA may be added to the physiological solution at a concentration of about 3-10 μg/mL, about 3-8 μg/mL, about 3-6 μg/mL, about 4-10 μg/mL, about 4-8 μg/mL, or about 4-6 μg/mL. In some embodiments, ASA may be added to the physiological solution at a concentration of about 4 μg/mL. In some embodiments, ASA may be added to the physiological solution at a concentration of about 5 μg/mL. In some embodiments, ASA may be added to the physiological solution at a concentration of about 6 μg/mL. In some embodiments, heparin or low molecular weight heparin (LWMH) may be added to the physiological solution at a concentration from may be from about 0.1 IU/mL to about 200 IU/mL or from about 0.5 IU/mL to about 150 IU/mL, or from about 1 IU/mL to about 140 IU/mL or from about 10 IU/mL to about 120 IU/mL or from about 30 IU/mL to about 100 IU/mL or from about 40 IU/mL to about 70 IU/mL or about 50 IU/mL or from 1 IU/mL to 100 IU/mL or from 5 IU/mL to 50 IU/mL or from 8 IU/mL to 20 IU/mL.

In another aspect, methods of preparing a personalized nerve graft can comprise contacting a surface of a nerve graft scaffold (e.g., an immunologically anonymized nerve segment, an artificial nerve graft) with an undiluted whole blood sample from a subject in need of the personalized nerve graft.

Contacting steps described herein with regard to methods of preparing a personalized nerve graft can be performed in a vessel. In some embodiments, the method further comprises coating the vessel containing the nerve graft scaffold with an anticoagulant prior to initiating the contacting. In some embodiments, the anticoagulant comprises heparin.

In some embodiments, certain components, such as for example, platelets, nucleated blood cells, and/or proteins, of a whole blood sample may be selected prior to the diluting.

In some embodiments, contacting is performed for at least 1 day, at least 2 days, at least 4 days, at least 8 days, at least 12 days, or at least 16 days. In some embodiments, contacting is performed for about 5-10 days. In some embodiments, contacting is performed for about 5 days. In some embodiments, contacting is performed for about 6 days. In some embodiments, contacting is performed for about 7 days. In some embodiments, contacting is performed for about 8 days. In some embodiments, contacting is performed for about 9 days. In some embodiments, contacting is performed for about 10 days. In some embodiments, contacting is performed at about room temperature (e.g., about 20-25° C.). In some embodiments, contacting is performed at about 30-40° C. (including, e.g., 30° C., 31° C., 32° C., 33° C., 34° C., 35° C., 36° C., 37° C., 38° C., 39° C., 40° C.). In some embodiments, contacting is performed at about 37° C. In some embodiments, contacting is performed with gentle shaking (e.g., using a shaker at about 25 rpm, 30 rpm, 35 rpm, 40 rpm, or 45 rpm).

Methods of preparing a personalized nerve graft can include obtaining blood (e.g., whole blood) from a subject in need of the personalized nerve graft. Whole blood for use in accordance with methods of preparing personalized nerve grafts described herein can be withdrawn into blood tubes, such as heparinized blood tubes. In some such embodiments, the heparinized blood tubes after addition of blood may yield a heparin concentration of about 5 IU/ml, about 10 IU/ml, about 17 IU/ml, about 20 IU/ml, about 30 IU/mL, about 40 IU/mL, about 60 IU/mL, about 80 IU/mL, about 100 IU/mL, about 150 IU/mL, about 200 IU/mL, or about 250 IU/mL. In some such embodiments, the heparinized blood tubes after addition of blood may yield a heparin concentration of about 17 IU/ml.

Blood (e.g., whole blood) for use in accordance with technologies of the present disclosure can be stored for a certain period of time at refrigerated temperatures (e.g., about 2 to about 6° C.), for example, prior to use in a method of preparing a personalized nerve graft described herein. In some embodiments, blood is stored for about 1 hour prior to use. In some embodiments, blood is stored for about 2 hours prior to use. In some embodiments, blood is stored for about 6 hours prior to use. In some embodiments, blood is stored for about 12 hours prior to use. In some embodiments, blood is stored for about 18 hours prior to use. In some embodiments, blood is stored for about 24 hours prior to use. In some embodiments, blood is stored for about 36 hours prior to use. In some embodiments, blood is stored for about 48 hours prior to use.

Without being bound by any theory, it may be understood that cells of blood (e.g., for use in accordance with technologies of the present disclosure) and non-cellular factors may populate a nerve graft scaffold. It may be understood that the one or more non-cellular factors of the blood may also populate the scaffold, and wherein the non-cellular factors may promote cellularization of the nerve graft scaffold and host compatibility of the nerve graft upon grafting. In some embodiments, whole blood samples for use in accordance with technologies described herein can comprise one or more non-cellular factors, wherein the one or more non-cellular factors of the whole blood can populate the scaffold, and wherein the non-cellular factors can promote cellularization of the nerve graft scaffold and host compatibility of the nerve graft upon grafting.

Furthermore, functionalization of anonymized nerve grafts can be achieved by impregnation of a nerve graft scaffold (e.g., an immunologically anonymized nerve segment) with certain components. Such components include, for example, certain extracellular vesicles, such as micelles, certain proteins, chemoattractants, and/or growth factors. Without wishing to be bound by any one theory, it is understood that such components can further increase the directed regrowth of axons through the nerve graft following implantation relative to an appropriate reference (e.g., an anonymized nerve graft not impregnated with certain components).

Blood (e.g., a suspension comprising whole blood, an undiluted whole blood sample) can further comprise an anti-thrombotic factor. In some embodiments, the anti-thrombotic factor comprises an anticoagulant agent, such as, for example, a heparin, a dextran, and/or acetylsalicylic acid.

In some embodiments, a concentration of the heparin in the suspension comprising the whole blood sample or in in the undiluted whole blood sample is from about 0.5 IU/mL to about 150 IU/mL at the beginning of contacting the surface of the nerve graft scaffold. In some embodiments, a concentration of the heparin in the suspension comprising the whole blood sample or in the undiluted whole blood sample is from about 30 IU/mL to about 100 IU/mL at the beginning of contacting the surface of the nerve graft scaffold. In some embodiments, a concentration of the heparin in the suspension comprising the whole blood sample or in the undiluted whole blood sample is about 50 IU/mL at the beginning of contacting the surface of the nerve graft scaffold.

In some embodiments, a concentration of the dextran in the suspension comprising the whole blood sample or in in the undiluted whole blood sample is from about 0.5 IU/mL to about 150 IU/mL or from about 0.5 IU/ml to about 80 IU/mL or from about 1 IU/ml to about 50 IU/mL or from about 5 IU/ml to about 40IU/mL or from about 10 IU/ml to about 30 IU/mL or about 20 IU/mL at the beginning of contacting the surface of the nerve graft scaffold. In some embodiments, a concentration of the dextran in the suspension comprising the whole blood sample or in the undiluted whole blood sample is from about 1 IU/mL to about 80 IU/mL or from about 5 IU/ml to about 40 IU/mL or from about 10 IU/ml to about 30 IU/mL or about 20 IU/mL at the beginning of contacting the surface of the nerve graft scaffold. In some embodiments, a concentration of the dextran in the suspension comprising the whole blood sample or in the undiluted whole blood sample is about 20 IU/mL at the beginning of contacting the surface of the nerve graft scaffold.

In some embodiments, a concentration of the acetylsalicylic acid in the suspension comprising the whole blood sample or in the undiluted whole blood sample is from about 0.2 μg/mL to about 200 μg/mL at the beginning of contacting the surface of the nerve graft scaffold. In some embodiments, a concentration of the acetylsalicylic acid in the suspension comprising the whole blood sample or in the undiluted whole blood sample is from about 3 μg/mL to about 10 μg/mL at the beginning of contacting the surface of the nerve graft scaffold. In some embodiments, a concentration of the acetylsalicylic acid in the suspension comprising the whole blood sample or in the undiluted whole blood sample is about 5 μg/mL at the beginning of contacting the surface of the nerve graft scaffold.

Additional reagents, such as, for example, growth factors, platelet-inhibitors, anticoagulants, can be added to the suspension comprising whole blood components or the undiluted whole blood sample.

In some embodiments, one or more growth factors are added to the suspension comprising whole blood components or the undiluted whole blood sample. Growth factors can include, for example and without limitation, a granulocyte macrophage-colony stimulating factor (GM-CSF), interleukin (IL)-3, IL-4, neutrophin (NT)-6, pleiotrophin (HB-GAM), midkine (MK), interferon inducible protein-10 (IP-10), platelet factor (PF)-4, monocyte chemotactic protein-1 (MCP-1), RANTES (CCL-5, chemokine (C-C motif) ligand 5), IL-8, IGFs, a fibroblast growth factor (FGF)-1, FGF-2, FGF-3, FGF-4, FGF-5, FGF-6, FGF-7, FGF-8, FGF-9, a transforming growth factor (TGF)-β, a vascular endothelial growth factor (VEGF), platelet-derived growth factor (PDGF)-A, PDGF-B, HB-EGF, hepatocyte growth factor (HGF), tumor necrosis factor (TNF)-α, insulin-like growth factor (IGF)-1, and any combination(s) thereof.

In some embodiments, the suspension comprising whole blood components or the undiluted whole blood sample further comprises equal to or more than the population average physiological level of one or more growth factors selected from the group consisting of: a granulocyte macrophage-colony stimulating factor (GM-CSF), interleukin (IL)-3, IL-4, neutrophin (NT)-6, pleiotrophin (HB-GAM), midkine (MK), interferon inducible protein-10 (IP-10), platelet factor (PF)-4, monocyte chemotactic protein-1 (MCP-1), RANTES (CCL-5, chemokine (C-C motif) ligand 5), IL-8, IGFs, a fibroblast growth factor (FGF)-1, FGF-2, FGF-3, FGF-4, FGF-5, FGF-6, FGF-7, FGF-8, FGF-9, a transforming growth factor (TGF)-β, a vascular endothelial growth factor (VEGF), platelet-derived growth factor (PDGF)-A, PDGF-B, HB-EGF, hepatocyte growth factor (HGF), tumor necrosis factor (TNF)-α, insulin-like growth factor (IGF)-1, and any combination(s) thereof.

In some embodiments, the one or more growth factors comprise a fibroblast growth factor-2. In some such embodiments, the fibroblast growth factor-2 is a recombinant human FGF-2 (rhFGF-2).

In some embodiments, the one or more growth factors comprise a vascular endothelial growth factor. In some such embodiments, the vascular endothelial growth factor is recombinant human VEGF (rhVEGF).

In some embodiments, the one or more growth factors comprise a fibroblast growth factor-2 (e.g., rhFGF-2) and a vascular endothelial growth factor (e.g., rhVEGF).

In some embodiments, the concentration of the fibroblast growth factor (e.g., rhFGF-2) in the undiluted whole blood sample is about 5 ng/mL, about 10 ng/ml, about 15 ng/ml, about 20 ng/ml, about 25 ng/mL, about 30 ng/ml, about 50 ng/mL, about 80 ng/mL, or about 100 ng/ml at the beginning of contacting the surface of the nerve graft scaffold. In some embodiments, the concentration of the fibroblast growth factor (e.g., rhFGF-2) in the undiluted whole blood sample about 5-30 ng/ml, about 5-25 ng/ml, about 5-20 ng/ml, about 5-15 ng/mL, about 1-100 ng/ml, about 1-80 ng/ml, about 1-50 ng/mL, about 1-30 ng/mL, or about 1-20 ng/mL at the beginning of contacting the surface of the nerve graft scaffold. In some embodiments, the concentration of the fibroblast growth factor (e.g., rhFGF-2) in the undiluted whole blood sample is about 5 ng/mL at the beginning of contacting the surface of the nerve graft scaffold. In some embodiments, the concentration of the fibroblast growth factor (e.g., rhFGF-2) in the undiluted whole blood sample is about 10 ng/ml at the beginning of contacting the surface of the nerve graft scaffold. In some embodiments, the concentration of the fibroblast growth factor (e.g., rhFGF-2) in the undiluted whole blood sample is about 15 ng/ml at the beginning of contacting the surface of the nerve graft scaffold.

In some embodiments, the concentration of the VEGF (e.g., rhVEGF) in the undiluted whole blood sample is about 1 ng/mL, about 10 ng/ml, about 20 ng/ml, about 30 ng/ml, about 40 ng/ml, about 50 ng/mL, about 60 ng/ml, about 70 ng/ml, about 80 ng/ml, about 90 ng/ml, about 100 ng/ml, about 110 ng/ml, about 120 ng/mL, about 130 ng/ml, about 140 ng/ml, about 150 ng/ml at the beginning of contacting the surface of the nerve graft scaffold. In some embodiments, the concentration of the VEGF (e.g., rhVEGF) in the undiluted whole blood sample is about 40-150 ng/ml, about 40-100 ng/mL, about 50-150 ng/mL, about 50-100 ng/mL, about 60-150 ng/mL, about 60-100 ng/mL, about 70-150 ng/mL, about 70-100 ng/ml, 1-150 ng/ml, about 1-100 ng/mL, about 10-150 ng/ml, about 10-100 ng/ml, about 20-150 ng/ml, about 20-100 ng/ml, about 30-150 ng/ml, or about 30-100 ng/ml at the beginning of contacting the surface of the nerve graft scaffold. In some embodiments, the concentration of the VEGF (e.g., rhVEGF) in the undiluted whole blood sample is about 70 ng/mL at the beginning of contacting the surface of the nerve graft scaffold. In some embodiments, vascular endothelial growth factor (e.g., rhVEGF) is added at a concentration of about 80 ng/ml at the beginning of contacting the surface of the nerve graft scaffold. In some embodiments, the concentration of the VEGF (e.g., rhVEGF) in the undiluted whole blood sample is about 90 ng/ml at the beginning of contacting the surface of the nerve graft scaffold.

In some embodiments, a method of preparing a personalized nerve graft is conducted one time. Methods of preparing personalized nerve grafts (e.g., as described herein) can be repeated. In some embodiments, a method of preparing a personalized nerve graft is conducted two times. In some embodiments, a method of preparing a personalized nerve graft is conducted three times. In some embodiments, a method of preparing a personalized nerve graft is conducted four times.

### Methods of Characterizing Personalized Nerve Grafts

A plurality of methods may be utilized to characterize a personalized nerve graft (e.g., a personalized nerve graft produced in accordance with technologies described herein). A personalized nerve graft may be characterized at any one or more points during a method of preparing a personalized nerve graft (e.g., as described herein). Alternatively, or additionally, a personalized nerve graft (e.g., prepared according to methods of the present disclosure) may be characterized after preparation, i.e. after the preparation is complete

A personalized nerve graft may be characterized, for example, by monitoring the concentration of D-glucose in the blood by measuring the concentration of D-glucose in a sample collected from the blood. In some embodiments, the D-glucose concentration is maintained between about 3 to about 11 mmol/L throughout the process. In order to maintain a D-glucose concentration between about 3 to about 11 mmol/L throughout the process, D-glucose may be supplemented to the blood as needed to compensate consumed D-glucose. In some embodiments, D-glucose may be measured every day of contacting.

A method of preparing a personalized nerve graft (e.g., as described herein) may further comprise measuring a concentration of D-glucose in the suspension and adding D-glucose to the suspension to maintain the concentration of D-glucose in the suspension between about 3 mmol/L and 11 mmol/L.

### Therapeutic Methods and Uses

In one aspect, the present disclosure provides methods of regenerating a nerve defect, comprising implanting an immunologically anonymized nerve graft prepared in accordance with technologies of the present disclosure.

In another aspect, the present disclosure provides methods of regenerating a nerve defect, comprising implanting a personalized nerve graft (e.g., prepared in accordance with technologies of the disclosure) to a subject, whose blood was used for preparing the personalized nerve graft.

Without being bound by any theory, it may be understood that nerve grafts described herein (e.g., immunologically anonymized nerve grafts, personalized nerve grafts) regenerate nerve defects, such as peripheral nerve defects, by providing biological guidance for re-growing axons. Thus, the nerve grafts described herein may be understood to function by providing an improved guiding structure, allowing the regenerating axons to find the correct path through the graft and into the nerve structure distally from the defect. This guidance enables the axons to reinnervate the target tissue after an appropriate time for regeneration (e.g., regrowth occurs at about 1 mm per day).

In some embodiments, the nerve defect may be a peripheral nerve defect. Peripheral nerve defects can range in severity and be classified into three general categories based on the presence of demyelination and the extent of damage of the axons and the connective tissues of the nerve. The mildest form of injury is called neurapraxia, defined by focal demyelination without damage to the axons or the connective tissues. Neurapraxia typically occurs from mild compression or traction of the nerve and results in a decrease in conduction velocity. Depending on the severity of demyelination, the effects may range from asynchronous conduction to conduction block, causing muscle weakness. The next level is called axonotmesis, which involves direct damage to the axons in addition to focal demyelination while maintaining continuity of the nerve's connective tissues. The most severe form of injury is called neurotmesis, which is a full transection of the axons and connective tissue layers wherein complete discontinuity of the nerve is observed. In some embodiments, a peripheral nerve defect is a neurapraxia nerve defect. In some embodiments, a peripheral nerve defect is an axonotmesis nerve defect. In some embodiments, a peripheral nerve defect is a neurotmesis nerve defect.

In some embodiments, a nerve defect (e.g., a neurotmesis nerve defect) may be of a certain length (e.g., distance in which full transection of the axons and connective tissue layers wherein complete discontinuity of the nerve is observed). In some such embodiments, a nerve defect may be about 1-10 cm long, about 2-9 cm long, about 2-8 cm long, about 3-9 cm long, about 3-8 cm long, about 4-8 cm long, about 5-8 cm long, or about 5-9 cm long. In some such embodiments, a nerve defect is at least 1 cm long, at least 2 cm long, at least 3 cm long, at least 4 cm long, at least 5 cm long, at least 6 cm long, at least 7 cm long, at least 8 cm long, at least 9 cm long, or at least 10 cm long. In some such embodiments, a nerve defect is about 4 cm long. In some such embodiments, a nerve defect is about 5 cm long. In some such embodiments, a nerve defect is about 6 cm long. In some such embodiments, a nerve defect is about 7 cm long. In some such embodiments, a nerve defect is about 8 cm long. In some such embodiments, a nerve defect is about 9 cm long. In some such embodiments, a nerve defect is about 10 cm long.

A plurality of surgical methods for implanting nerve grafts are known in the art. Nerve grafts or nerve graft scaffolds of the present disclosure (e.g., immunologically anonymized nerve grafts, personalized nerve grafts) can be implanted according to any suitable method known in the art and one of ordinary skill in the art could readily select and use such implantation methods in accordance with the technologies of the present disclosure. In some embodiments, damaged ends of a nerve defect (e.g., on or both sides of the damaged area) are resected and/or damaged tissue is removed prior to surgically implanting a nerve graft of the present disclosure. In some embodiments, a nerve graft of the present disclosure bridges a transected nerve defect.

The effectiveness of methods of regenerating a nerve defect can be assessed by evaluating a parameter (e.g., nerve conduction, muscle function) before and after methods of regenerating a nerve defect described herein are conducted. Any assay known in the art can be used to evaluate the effectiveness of the methods described herein. Such assays include, for example and without limitation, electrophysiological assays (e.g., nerve conduction tests, electromyographic (EMG) monitoring), ultrasound examinations (e.g., to measure cross-sectional area and/or perimeter of certain muscles), and histological assays (e.g., measuring the cross-section area of the nerves and grafts, counting the number of NF200 labeled axons in systematically selected fields, counting the total number of regenerated myelinated axons).

### Patient Populations

In some embodiments, a subject in need thereof in accordance with the technologies described herein include, but are not limited to, humans and non-human vertebrates. In some embodiments, a subject in need thereof in accordance with technologies described herein is, for example, a mammal. Mammals can include, for example and without limitation, a household pet (e.g., a dog, a cat, a rabbit, a hamster), a livestock or farm animal (e.g., a cow, a pig, a sheep, a goat, a chicken), a horse, a non-human primate, and a laboratory animal (e.g., a mouse, a rat, a rabbit). In a preferred embodiment, the subject in need thereof in accordance with technologies of the present disclosure is a human, a livestock or farm animal, or a horse. In some embodiments, the subject in need thereof in accordance with technologies of the present disclosure is a human.

In some embodiments, the subject in need thereof is an adult. In some embodiments, the subject in need thereof is a human subject over 18 years of age. In some embodiments, the subject in need thereof is a human subject over 21 years of age. In some embodiments, the subject in need thereof is a human subject over 30 years of age. In some embodiments, the subject in need thereof is a human subject over 65 years of age. In some embodiments, the subject in need thereof is a human subject under 18 years of age. In some embodiments, the subject in need thereof is a human subject under 65 years of age, between 18 and 65 years of age, between 21 and 65 years of age, or between 30 and 65 years of age. In some embodiments, the subject in need thereof is a human subject under 12 years of age (e.g., a pediatric subject).

In some aspects, technologies of the present disclosure can be practiced in any subject that has (e.g., that has been diagnosed with) a nerve defect. In some embodiments, a nerve defect is a peripheral nerve defect. In some embodiments, a peripheral nerve defect is a neurapraxia nerve defect. In some embodiments, a peripheral nerve defect is an axonotmesis nerve defect. In some embodiments, a peripheral nerve defect is a neurotmesis nerve defect.

In some embodiments, technologies of the present disclosure can be practiced in a subject that has a plurality of nerve defects.

Tests for diagnosing nerve defects to be regenerated by technologies described herein are known in the art and can be readily understood and utilized by the ordinary medical practitioner. Such tests include, for example and without limitation, magnetic resonance imaging (MRI), computed tomography (CT) scan, electromyography (EMG), ultrasound, and nerve conduction studies. Generally, medical practitioners also take a full medical history and conducts a complete physical examination in addition to the tests listed above.

### Combination Therapies

In some aspects, technologies of the present disclosure methods of regenerating a nerve defect further comprising use of additional agents and/or therapies that can enhance effectiveness of methods described herein (e.g., methods for regenerating a nerve defect), such as, for example, enhance regeneration, improve nerve conduction, reduce pain, and/or improve motor skills associated with nerve defect.

In some embodiments, methods of regenerating a nerve defect described herein further comprises administering a subject in need thereof one or more analgesic, including, for example, analgesics for peripheral neuropathy. Examples of analgesics include, without limitation, aspirin, ibuprofen, corticosteroid injections, amitriptyline, duloxetine, pregabalin, and gabapentin.

In some embodiments, methods of regenerating a nerve defect described herein further comprises the use of braces and/or splints, electrical stimulation therapy (e.g., to activate muscles served by an injured nerve while the nerve regenerates), physical therapy, and/or exercise.

In certain embodiments, additional agents and/or therapies can be administered before, during (e.g., concurrently), and/or after methods of regenerating a nerve defect described herein.

### Kits

In one aspect, the present disclosure provides kits comprising (i) materials for non-chemically removing cells from a nerve segment; (ii) materials for enzymatically removing immunogenic remnants from the nerve segment to provide an immunologically anonymized nerve segment; (iii) optionally, instructions for preparing an immunologically anonymized nerve segment.

In another aspect, the present disclosure provides kits comprising (i) one or more containers; (ii) materials for obtaining blood from a subject; and (iii) optionally, instructions for preparing a personalized nerve graft.

A container may include, for example and without limitation, a vial, well, test tube, flask, bottle, syringe, infusion bag, or other container means. Where an additional component is included in the kit, the kit can include additional containers into which this component may be placed. Containers and/or kits can comprise labeling with instructions for use and/or warnings.

### EXAMPLES

### Example 1: Materials and Methods

The following materials and methods were used for the following Examples.

Immunological anonymization of nerve segments: Rat sciatic nerves were harvested from euthanized Sprague Dawley rats under aseptic conditions. Specimens of sheep nerves for obtaining the immunologically anonymized allografts were provided from the colony of Servei de Granges i Camps Experimentals (SGiCE) of the Universitat Autònoma de Barcelona (UAB). Peroneal nerves were harvested from euthanized sheep under aseptic conditions. The harvested nerves were stored in phosphate buffered saline (PBS) plus antibiotic-antimycotic agents, maintained at 4° C. and shipped to Verigraft AB.

For removal of immunogenic components, the nerve segments were thawed and agitated in NaCl (Fisher Scientific) for 24 hours, PBS (Medicago) for 6 hours and Deoxyribonuclease (DNase; VWR) overnight. The nerves were washed 3×5 min in H₂O after NaCl and DNase. This process was repeated three times. Then the nerves were washed in PBS for 24 hours. After removal of immunogenic components, samples were taken for DNA quantification and histology before peracetic acid sterilization and final washes in PBS under sterile conditions. The sterilized immunologically anonymized nerve grafts were shipped in PBS at 2-4° C. One nerve segment was immunologically anonymized for each animal to be transplanted. DNA was extracted from 10-25 mg wet tissue with the DNeasy Blood & Tissue kit (Qiagen) and quantified with the Qubit dsDNA HS assay kit (Life Technologies), according to the manufacturer's instructions.

After removal of immunogenic components, a small segment of each immunologically anonymized nerve graft was fixed in paraformaldehyde 4% for 4 hours at room temperature (RT) and then transferred to PBS with sucrose 30% for 24 hours. Samples were cryo-embedded with OCT and 15 μm thick transverse sections were obtained with a cryotome. The sections were processed for immunohistochemistry with primary antibodies against S-100 protein (S100; Schwann cells; 1:50; 22520-DiaSorin), neurofilament (NF200; myelinated axons; 1:400; AB5539-Millipore), non-collagenous connective tissue glycoprotein (laminin; 1:500; AHP420-Biorad), and with myelin stain (Fluoromyelin; 1:300; F34651-Invitrogen) and with DAPI staining for nuclei (1:100; D9564-10 MG-Sigma). Following washes, the sections were incubated with secondary antibodies conjugated to Alexa Fluor 488 goat anti-chicken (1:200; A11039-Invitrogen) and Alexa Fluor 594 goat anti-rabbit (1:200; A21207-Invitrogen). Immunolabeled sections were viewed under epifluorescence microscopy (Olympus BX51).

Rat study: All procedures were approved by the Ethics Committee of the Universitat Autònoma de Barcelona and Generalitat de Catalunya, and followed the European Community Council Directive 2010/63/EU of the European Parliament on the protection of animals used for scientific purposes.

For the implantation, ten female Sprague Dawley rats (12 weeks of age) were randomly assigned into two different experimental groups: autograft (AG) (n=5) or immunologically anonymized rat allograft (DRA) (n=5). Animals were anesthetized with ketamine (75 mg/kg) and medetomidine (0.01 mg/kg) intraperitoneally. A skin incision was made, and the right sciatic nerve was exposed by splitting the biceps femoris muscle. A 15 mm long nerve segment was excised and then, in the AG group was sutured again whereas in the DRA group it was substituted by a 15 mm long immunologically anonymized rat allograft. The proximal and distal nerve stumps were sutured by 10-0 nylon epineural sutures. Postoperative care included amytriptilin (20 ml/l) in drinking water to prevent autotomy (Navarro et al., 1994) and buprenorphine (0.03 mg/kg subcutaneously) to treat postoperative pain. Reinnervation of target muscles were assessed monthly until the end time, set at 120 days post injury (dpi). Under general anesthesia, the sciatic nerve was stimulated at the sciatic notch and the compound muscle action potential (CMAP) was recorded from tibialis anterior (TA), gastrocnemius (GM) and plantar interosseus (PL) muscles, using a Sapphyre 4ME electromyography (EMG) device (Vickers Healthcare Co).

At the end of the follow-up, animals were euthanized by an overdose of pentobarbital (200 mg/kg i.p.). Sciatic nerves were harvested and fixed in paraformaldehyde 4% and then divided in three different parts. The proximal and distal segments, including suture levels were post-fixed in 3% glutaraldehyde-3% paraformaldehyde in cacodylate-buffer solution (0.1 M, pH 7.4) at 4° C. To evaluate the microstructure of the nerve, samples were embedded in epon resin. Semithin 0.5 mm sections were stained with toluidine blue and sets of images obtained at 400× and 1000× with a light microscope (Olympus BX40) were chosen to measure the cross-sectional area and counts of the number of myelinated axons with ImageJ software. The middle segment of the sciatic nerve was processed for immunolabeling of axons (NF200), Schwann cells (S100), macrophages (ionized calcium-binding adapter molecule 1 (Iba1); 1:500; 19-19741-Rafer) and laminin. Samples were washed and incubated with secondary antibodies Alexa Fluor 488 Goat antichicken (1:200; A11039-Invitrogen), Alexa Fluor 488 Donkey anti-goat (1:200; A11055-Invitrogen) and Alexa Fluor 594 Goat anti-rabbit (1:200; A21207-Invitrogen) diluted in PBS-Triton 0.3%. Finally, sections were cover-slipped with Fluoromount containing DAPI (1:10000; Sigma-Aldrich). Sections were visualized with an epifluorescence microscope (Olympus BX51).

Sheep study: Ten female ripollesa sheep (Ovis aries), weighing 55-75 Kg, from the animal facility of Servei de Granges i Camps Experimentals (SGiCE) of the Universitat Autònoma de Barcelona were used. The sheep were divided in two experimental groups of 5 animals each, according to the repair procedure performed: autograft and immunologically anonymized allograft. A blood sample was taken before the surgery and at the end of the study to perform hematological and biochemical standard analyses, to ensure that the sheep had normal profile. General clinical assessment was performed once a week before the surgery until the end of the study after 9 months.

Surgical procedure: The animals were fasted 16 hours prior to the surgery to reduce the ruminal content and prevent deviant swallowing. Animals were sedated by midazolam (0.2 mg/Kg) and morphine (0.4 mg/Kg) intramuscularly and anesthesia was induced by intravenous propofol (4 mg/Kg) and maintained by isofluorane 2% in 2 L/min oxygen administered with a pressure-controlled ventilator. Analgesia was provided by diazepam (0.5 mg/Kg) intravenously, and an antibiotic dose of cefazoline (20 mg/Kg) was given intravenously. Fluid therapy was applied with Ringer solution infusion (10 mL/Kg/h).

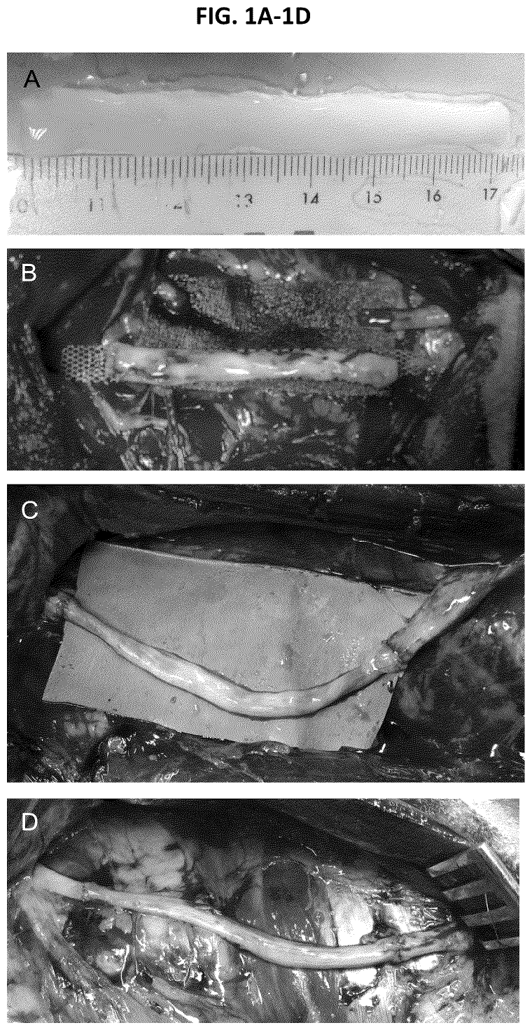

The operative procedure was carried out using a sterile technique with the sheep in a left lateral decubitus position on an operating table. On the right hindlimb, the peroneal nerve was exposed using a longitudinal lateral skin incision along the thigh followed by splitting of the semitendinous and biceps femoris muscles. Under the operating microscope, the common peroneal nerve was resected 1 cm above the iliac vein to create a 7 cm gap. The repair was carried out by bridging the two nerve stumps with an immunologically anonymized nerve allograft or with an autograft (FIG. 1A-1D). The immunologically anonymized nerve graft was easy to handle and suture, without notable difference compared with the nerve autograft. The nerve stumps were sutured to each end of the graft by means of 8/0 epineural sutures. The suture resistance was tested against light stretching. The incision was closed in layers and disinfected with chlorhexidine and Aluspray®. After the surgery the animals were allowed to recover in couples in the animal facility. During the post-operative period, analgesia was provided twice a day with buprenorphine (0.01 mg/Kg s.c.) for 2 days and once a day with meloxicam (0.2 mg/Kg s.c.) for 3 days.

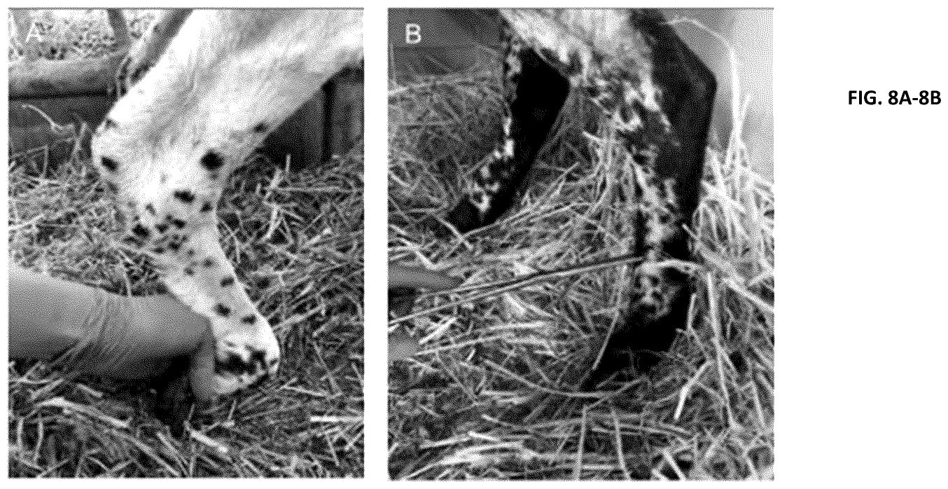

Functional tests: At monthly intervals, animals were tested for evidence of hindlimb functional recovery. Each parameter was assessed in an arbitrary scale from 0 (no deficit) to 2 (complete loss). Locomotion was assessed while freely walking in the stable, paying particular attention to the foot-drop position of the operated hindlimb. The tibialis anterior (TA) muscle mass was assessed by palpation comparing both denervated and control side. The proprioceptive response was tested by the capability of replacing the hindpaw from the plantarflexion position to a plantar support position. The withdrawal reflex of the paw was evaluated by pinching with a hemostat the skin of the dorsum of the paw at three sites, proximal, middle and distal (FIG. 8A-8B).

Electrophysiological tests: Motor nerve conduction tests were performed at 6.5 and 9 months after the operation under general anesthesia (diazepam 0.25 mg/Kg and ketamine 5 mg/Kg i.v.). An EMG apparatus (Sapphyre 4ME, Vickers Healthcare Co, UK) was used for these tests. The sciatic nerve was stimulated at the sciatic notch and the knee with percutaneous needle electrodes and the compound muscle action potential (CMAP) was recorded from the tibialis anterior (TA) muscle with monopolar needle electrodes. Free-running EMG recordings were made to detect fibrillation potentials, as a sign of muscle denervation.

Ecographic evaluation of hindlimb muscles: Ultrasound examinations were performed using a MyLab® Gamma (Esaote. Italy) device and a linear ultrasound probe. The cross-sectional area and perimeter of the TA muscle was determined using B-mode ultrasound, employing a 15 MHz linear transducer and optimizing the image at a depth of 3 cm and focal point at 1.5 cm. To optimize image acquisition and good skin contact, the hair over the area of interest was clipped and the skin cleaned with water and mild soap, and acoustic gel was used. In preliminary studies, it was established that the point of interest to standardize image acquisition was the cranial aspect of the crus at a midpoint between the tibial crest and the tuber calcani.

Histological evaluation: After performing the functional test 9 months after injury and when still anesthetized, animals were euthanized using an intravenous injection of Euthasol (400 mg/Kg i.v.). Nerve segments extending at least 1 cm from the proximal suture and 2 cm from the distal suture of the graft, were harvested and fixed in paraformaldehyde 4% for 7 days at 4° C. TA muscles were taken and weighed, and samples of TA muscles and of the skin of the dorsum of the paw were obtained and fixed in formol 4% for 7 days at room temperature.

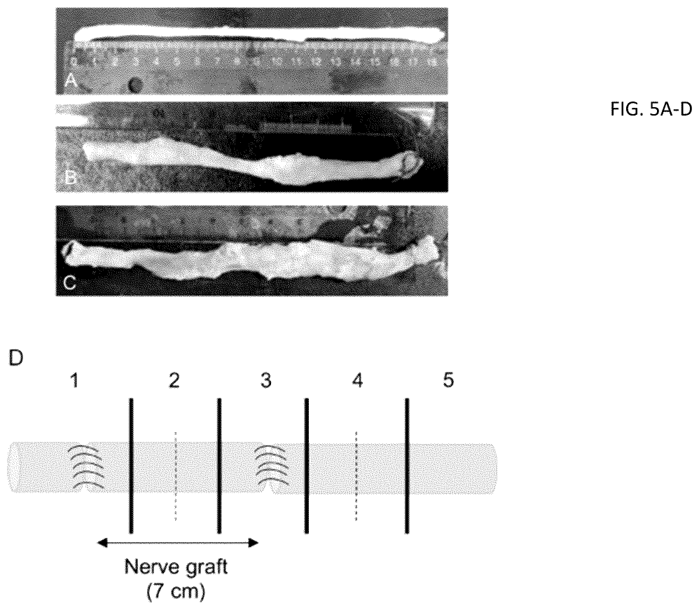

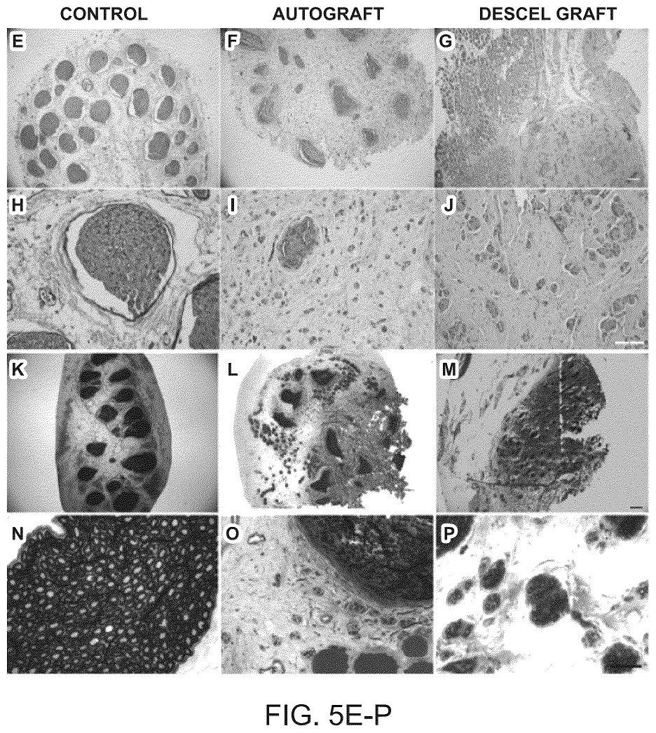

After fixation, the peroneal nerves were divided into 5 different sections (FIG. 5D), and each section was further divided in two halves. The first half of section 2 from the middle of the graft and section 4 from the distal end were embedded in paraffin and 5 μm thick cross sections were obtained with a microtome. Samples from the midpoint of the graft were deparaffinated and stained with hematoxylin and eosin to visualize the general structure of the nerve. Other sections were dewaxed and processed for immunohistochemistry against S-100 protein to label Schwann cells, and neurofilament NF200 to label myelinated axons. Following washes, the sections were incubated with secondary antibodies bound to Alexa Fluor 488 and Alexa Fluor 594. Sections were finally mounted on gelatinized slides and viewed under epifluorescence microscopy. Quantitative analysis was performed by measuring the cross-section area of the nerves and grafts and counting the number of NF200 labeled axons in systematically selected fields; then, the total number of regenerated myelinated axons was estimated. The second half of section 2 from the middle of the graft and section 4 from the distal nerve were postfixed in 3% paraformaldehyde and 3% glutaraldehyde in phosphate-buffered saline. The nerve segments were postfixed in osmium tetroxide, dehydrated in series of ethanol, and embedded in Epon resin. Semithin 0.5 μm thick sections were stained with toluidine blue, and representative images were taken by light microscopy.

TA muscle and skin samples were also processed for paraffin embedding. Samples were stained with hematoxylin and eosin to visualize the general structure and to evaluate the degree of atrophy.

Data analysis: Data are expressed as mean±standard error of the mean (SEM). The results of functional tests and histology were analyzed by Student's t test and two-way ANOVA after checking for normal distribution, using GraphPad Prism 8 software. A p<0.05 was considered as significant.

### Example 2: Effectiveness of the Procedure of Immunological Anonymization

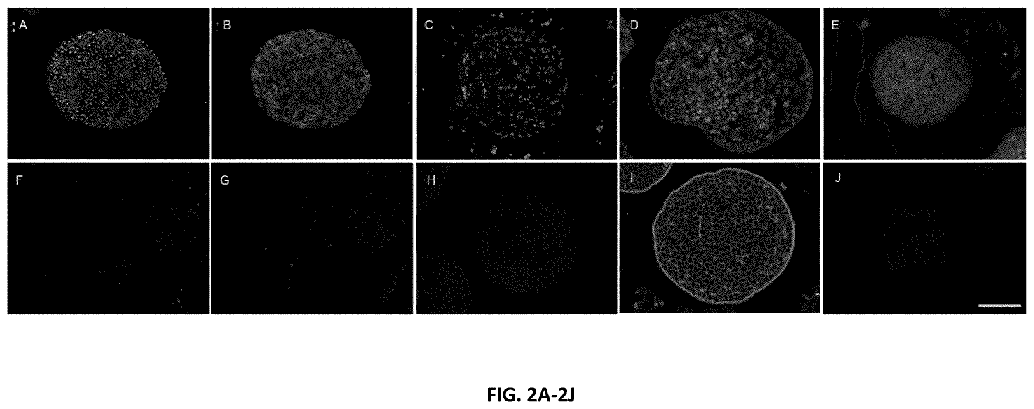

The present example demonstrates, among other things, effectiveness of procedures of immunological anonymization described herein. The procedure of immunological anonymization developed removed most or all donor cells and DNA from the nerve segment, while the Extracellular Matrix (ECM) as well as structural layers of the graft remained intact. Without wishing to be bound by any one theory, it is understood that, in this way, the nerve grafts can provide guidance to the regenerating axons upon implantation. Cross sections of immunologically anonymized nerve grafts were processed for immunohistochemistry before implantation. Immunolabeling against laminin showed that the structure of the ECM was well preserved. Faint staining against NF200, S100 and fluoromyelin in the immunologically anonymized peroneal nerve grafts was compatible with lack of axons (NF200) and Schwann cells (S100), and good decellularization (absence of DAPI, general marker of nucleus), thus indicating that cell contents were completely or nearly completely removed (FIG. 2A-2J).

### Example 3: Rat Study

The present example demonstrates assessment of TA, GM, and PL muscles one month after surgery.

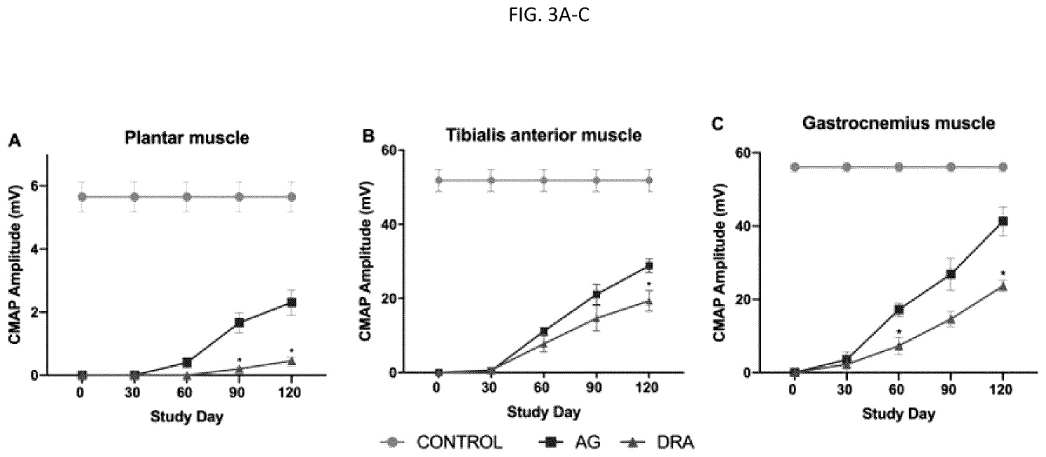

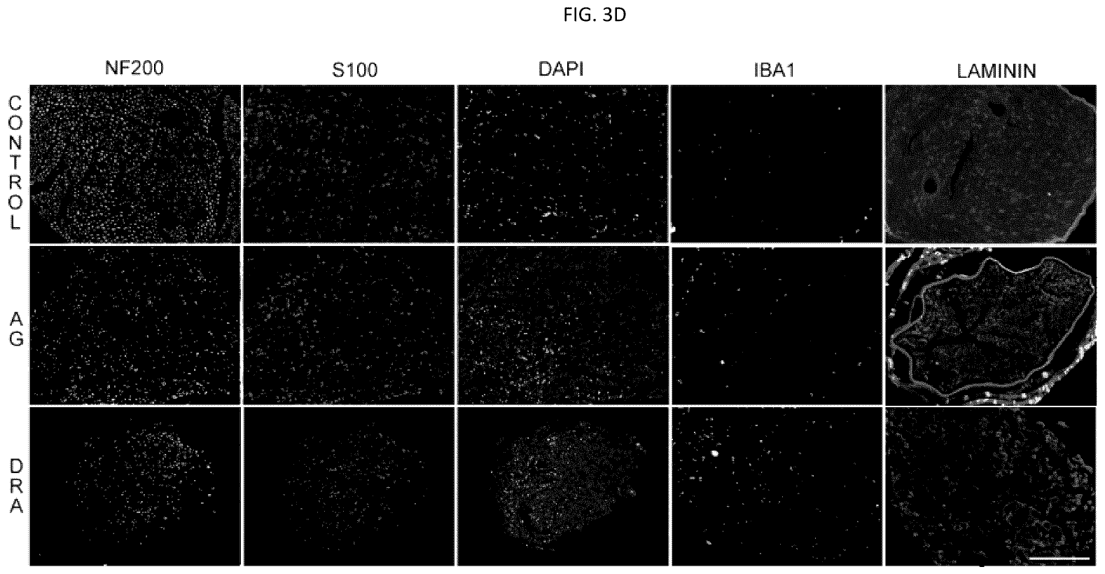

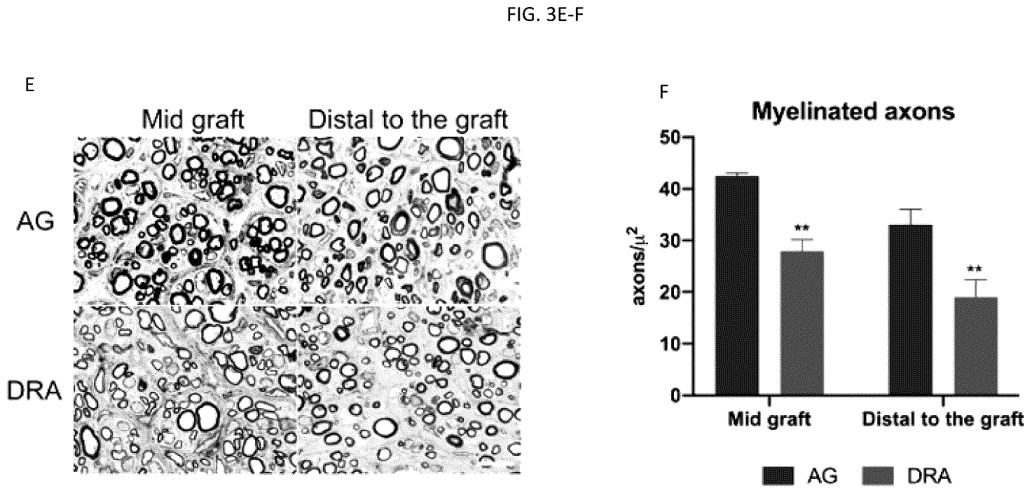

Electrophysiological tests showed complete or nearly complete denervation of TA, GM and PL muscles one month after the surgery. At 60 dpi all animals from AG and DRA groups showed evidence of starting reinnervation in the TA and GM muscles. Three of the animals of the AG group showed positive values whereas none of the animals of the DRA group showed evidence of reinnervation in the PL muscle. At 90 dpi, the amplitude of the CMAPs increased for TA and GM muscles in both groups. All the rats of the AG group but only two thirds of the rats of the DRA group showed positive values for PL muscle. At the end of the follow-up, set up at 120 dpi, group AG had significantly higher mean CMAP amplitude values than the DRA group in TA (28.8 [symbol] 1.8 mV vs 19.3 [symbol] 2.8 mV; p<0.05), GM (41.3 [symbol] 3.9 mV vs 23.7 [symbol] 1.5 mV; p<0.05) and PL (2.3 [symbol] 0.4 mV vs 0.5 [symbol] 0.1 mV; p<0.05) muscles (FIG. 3A-C).

The histological observations of transverse nerve sections showed the typical appearance of regenerated nerves, with numerous small size myelinated and unmyelinated axons inside the endoneurial compartments of the nerve graft and in the distal nerve. Immunohistochemical labeling showed numerous axons accompanied by Schwann cells along the grafts, but at lower density than in control nerves (FIG. 3D). Quantitative analysis of the myelinated axons showed significantly higher density in the middle of the graft in the AG group (42.5 0.6 axons/mm²) compared to DRA group (33.1 [symbol] 2.9 axons/mm²; p<0.01 vs AG). Distally to the graft, all animals from AG and DRA groups had regenerated myelinated axons, but their density was significantly higher in the AG group (27.9 [symbol] [symbol] 02.2 axons/mm²) than in the DRA group (19.0 [symbol] [symbol] 3.4 axons/mm²) (FIG. 3E-F).

### Example 4: Sheep Study

The present example demonstrates results using nerve graft methods described herein on sheep.

All sheep from both groups recovered from the surgical procedure without any side effect and survived until the end of the study. No significant clinical signs, except for the right hindlimb dysfunction, were observed in the animals during the experimental follow-up. The sheep were able to stand and walk and have good mobility. As expected, due to the peroneal nerve injury, two animals had a marked foot-drop posture that produced pressure ulcers on the dorsal skin of the paw. A splint was placed 30 days post-operation to prevent further foot lesions, and ulcers were treated with chlorhexidine and antibiotic cream and covered. Only one of the sheep did not recover and continued with the foot-drop posture until the end of the study.

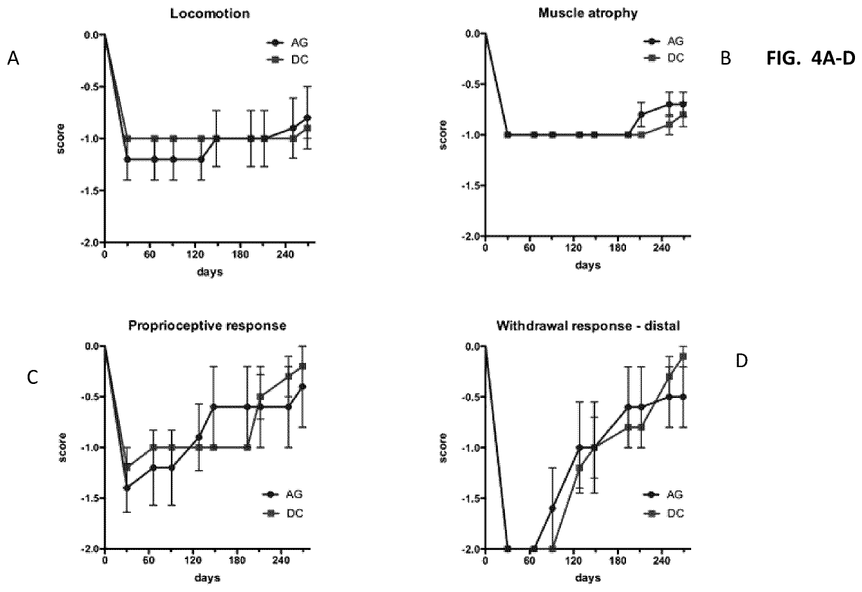

All the sheep showed close to normal locomotion activity after the surgery. In the resting orthostatic position, all the sheep, except one from AG group, were able to correctly position the right hind hoof, maintaining the plantar support. When tested during fast walking, a foot-drop gait was evidenced in all sheep, as they failed to make plantar stepping in some steps (scored 1-2). A slight improvement in dorsiflexion was observed in the last 2 months of the follow-up (FIG. 4A-D). The right TA muscle showed a reduction in size from one month after the surgery. There was some mild improvement appreciated in 3 animals of the AG group and in 2 of the DC group during the last 2 months (FIG. 4A-D). The reposition of the injured hindlimb when displaced to the dorsum, indicating proprioceptive inputs, was partially reduced after the surgery and the score recovered to normal value with time. The withdrawal reflex response was induced in 3 different sites, proximal, mid and distal in the dorsum of the paw. The response was suppressed after the surgery and slowly recovered with time. At the distal site, the test was more sensitive, without full recovery in all the sheep at 9 months post-operation (FIG. 4A-D).

Overall, the functional tests showed the expected failure due to a peroneal nerve injury, and partial recovery of sensory-mediated functions, whereas motor recovery and muscle mass remained relatively steady until the last month of follow-up.

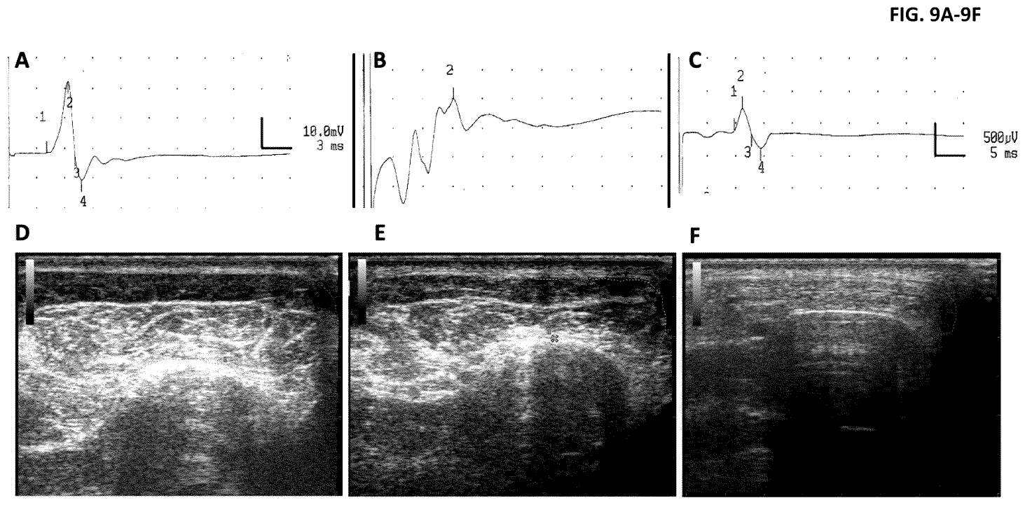

In the left control side, the TA CMAP, evoked by stimulation at the sciatic notch, appeared at an onset latency of 4.2-4.4 msec and had a maximal amplitude of 20-22 mV, with small variations between sheep (FIG. 9A-9F). In the right, operated side, at 6.5 months CMAPs of late latency, small amplitude and disperse shape were recorded in 3 sheep of group AG and in 3 sheep of group DC. Fibrillation potentials, sign of existing muscle denervation, were detected in muscles without CMAP response. At 9 months, CMAPs, recorded in 4 sheep of each group, were of slightly higher amplitude and still of long latency, compatible with early reinnervation of the TA muscle. No significant differences were found between groups AG and DC in any parameter of the nerve conduction tests (Table 1).

In denervated muscles, the density of the muscle imaging changed, and the size (measured both as area and perimeter) was decreased, evidencing muscle atrophy and increased connective tissue secondary to denervation (FIG. 9A-9F). At 6.5 months post-surgery, the TA muscle area in the DC group was significantly lower than in the AG group (p<0.01). At the end of the follow-up, there was a tendency to recover muscle size, although it was still significantly lower in the DC group than in the AG group (p<0.05) (Table 1). The size of the TA muscles in the two sheep without electrophysiological responses was the smallest, pointing up a relationship between muscle size and degree of reinnervation.

TABLE 1
Values of the nerve conduction tests in the peroneal nerve and of
the echography tests in the peroneal innervated muscles,
in the control left limb and in the operated right limb
at 6.5 and 9 months postoperation.

| | | | CMAP Amplitude | N + |
|---|---|---|---|---|
| | Group | Latency (msec) | (mV) | response |
| Nerve conduction | | | | |
| Control (L) | AG | 4.38 ± 0.16 | 21.26 ± 1.54 | 5/5 |
| Control (L) | DC | 4.24 ± 0.09 | 22.84 ± 1.02 | 5/5 |
| 6.5 mo | AG | 11.05 ± 1.52 | 0.97 ± 0.48 | 3/5 |
| 6.5 mo | DC | 16.87 ± 4.54 | 0.21 ± 0.11 | 3/5 |
| 9 mo | AG | 11.58 ± 1.06 | 1.87 ± 0.72 | 4/5 |
| 9 mo | DC | 13.16 ± 1.50 | 0.46 ± 0.16 | 4/5 |
| | | TA area (cm²) | TA perimeter (cm) | |
| Echography | | | | |
| Control (L) | AG | 5.30 ± 0.29 | 10.30 ± 0.24 | |
| Control (L) | DC | 5.31 ± 0.48 | 10.30 ± 0.44 | |
| 6.5 mo | AG | 1.90 ± 0.07 | 7.34 ± 0.23 | |
| 6.5 mo | DC | 1.48 ± 0.03** | 6.87 ± 0.08 | |
| 9 mo | AG | 2.44 ± 0.21 | 7.95 ± 0.17 | |
| 9 mo | DC | 1.85 ± 0.09* | 7.45 ± 0.11 | |

*p < 0.05 vs AG;
**p < 0.01 vs AG.

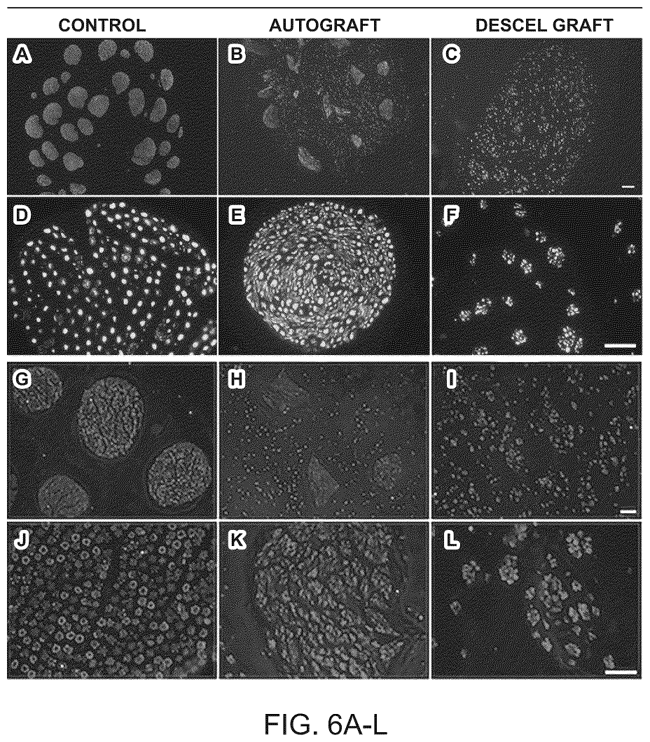

Most of the animals from AG and DC groups presented grafted nerves with a well-preserved structure and neuroma visible at proximal and distal ends corresponding with the suture lines (FIG. 5A-5P). The control peroneal nerve was composed of multiple small fascicles, usually more than 30, each containing numerous nerve fibers densely packed in the endoneurium. In the AG group, the structure of the fascicles was preserved in the graft, but regenerating axons were detected both inside and outside of the perineurium delimiting the fascicles. In contrast, no clear fascicles were observed in the immunologically anonymized grafts, where regenerating axons were seen grouped in small regenerative units, disperse within the graft structure (FIG. 5A-5P). Distal to the grafts, the multifascicular structure of the peroneal nerve was maintained and most regenerating axons grew within the distal nerve fascicles. Immunohistochemical labeling at the middle part of the grafts from AG animals showed axons and Schwann cells (FIG. 6A-6L) present within both fascicular and extra-fascicular areas, whereas in the immunologically anonymized grafts of the DC group, axons and glial cells were dispersed in small regenerative clusters. Regarding the quantitative analysis of NF200 labeled axons, group AG had 1 animal with a very low number of regenerating axons while 2 animals had higher than control values. One animal did not show regenerated axons, likely attributable to suture dehiscence early after the operation. Group DC also had 1 animal without regenerating axons. The estimated mean number of myelinated axons was 21,676±3,716 in the control peroneal nerve, whereas in the mid segment of the grafts there were 26,213±2,798 axons in the four regenerated sheep of the AG group, and 18,724±3,410 in the four sheep of the DC group. The corresponding values at the nerve distal to the graft were 20,600±6,082 for the AG group and 7,780±1,871 (p<0.05) for the DC group. The higher number of regrowing axons in the graft is understood to be indicative of some excessive regenerative sprouting that was remodeled at the distal nerve.

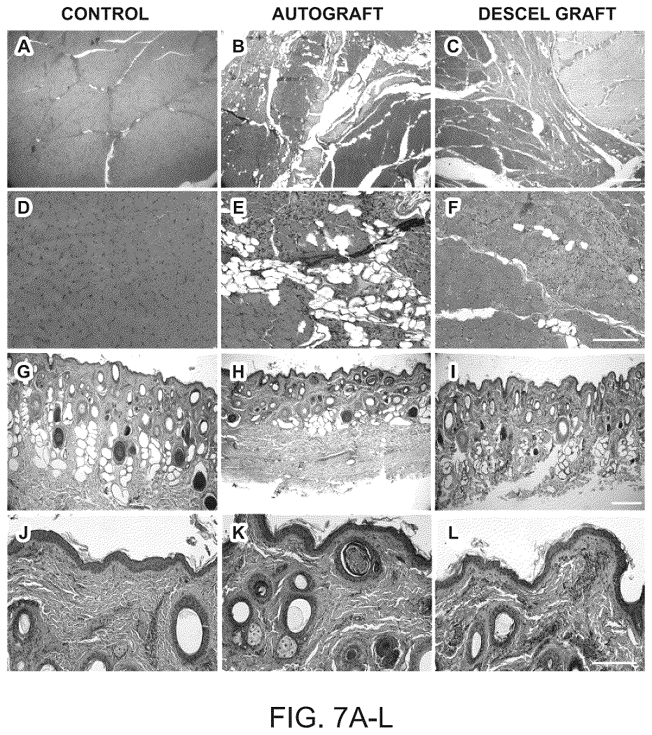

Semithin sections stained with toluidine blue showed in the AG group preserved structure of the fascicles with a large number of myelinated axons of different sizes and in addition, small new regenerative units that contained small number of myelinated axons. In the DC group, the structure of the fascicles was not conserved, although new regenerative units were observed throughout the nerve with a large number of myelinated axons (FIG. 7A-7L).

In the operated hindlimb, the TA muscle showed a heterogeneous structure in both groups, AG and DC (FIG. 7A-L). Some areas appeared with a normal appearance, although muscle fibers had smaller diameter than normal, likely corresponding to reinnervated areas of the muscle. Other areas showed signs of atrophy and inflammatory infiltration. The skin samples, processed in sagittal sections, showed distinct organization in three layers: epidermis, dermis and subcutaneous tissue. The skin of the dorsum of the paw had a similar aspect to the skin from the contralateral side, without signs of cell infiltration or atrophy (FIG. 7A-L).

### Example 5 DNA Quantification and Cell Detection in Immunologically Anonymized Nerve Grafts

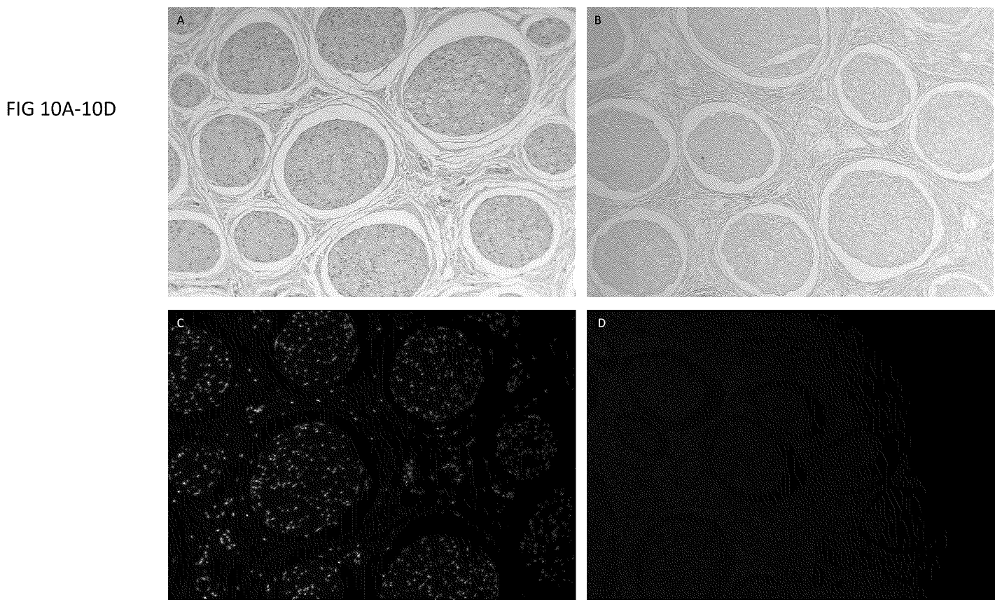

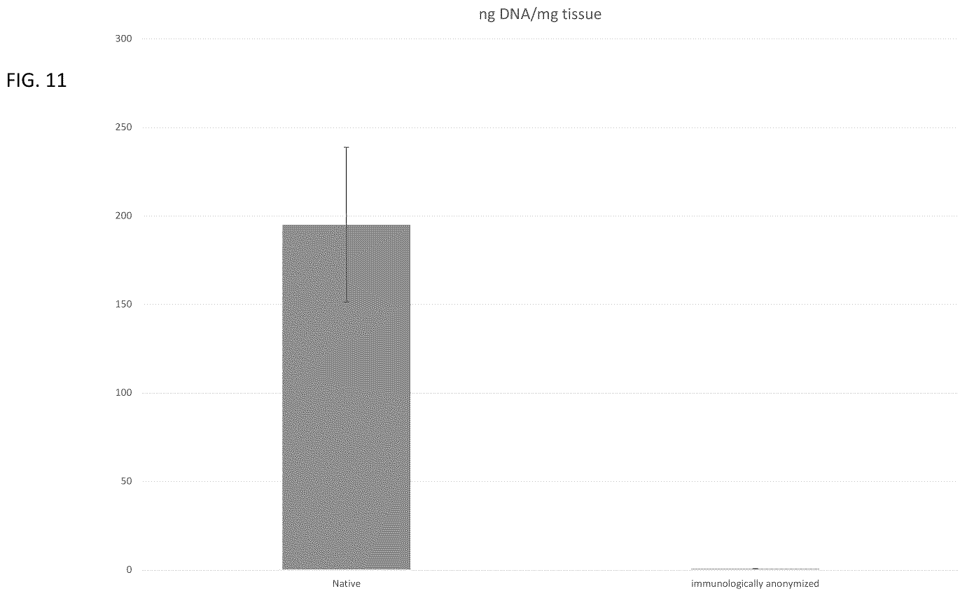

After processing, immunologically anonymized nerve grafts were analyzed both using histochemical staining (FIG. 10A-10D) of cell nuclei and DNA quantification (FIG. 11) to confirm successful removal of immunogenic components from explanted nerve segments.

FIG. 10A-10D, evaluation of the removal of cellular components. 4,6-diamidino-2-phenylindole (DAPI) and Hematoxylin-Eosin (H&E) staining was performed on cross-sections of 5 μm thickness, following standard protocols. Images were acquired using a fluorescence microscope (Zeiss Axiovert 40 CFL) for DAPI visualization, and in bright field for H&E visualization.

FIG. 11: evaluation of DNA removal. DNA was extracted from 10-25 mg wet tissue with the DNeasy Blood & Tissue kit (Qiagen) and quantified with the Qubit dsDNA HS assay kit (Life Technologies), according to the manufacturer's instructions.

### Example 6: DNA Quantification and Cell Detection in Personalized Nerve Grafts

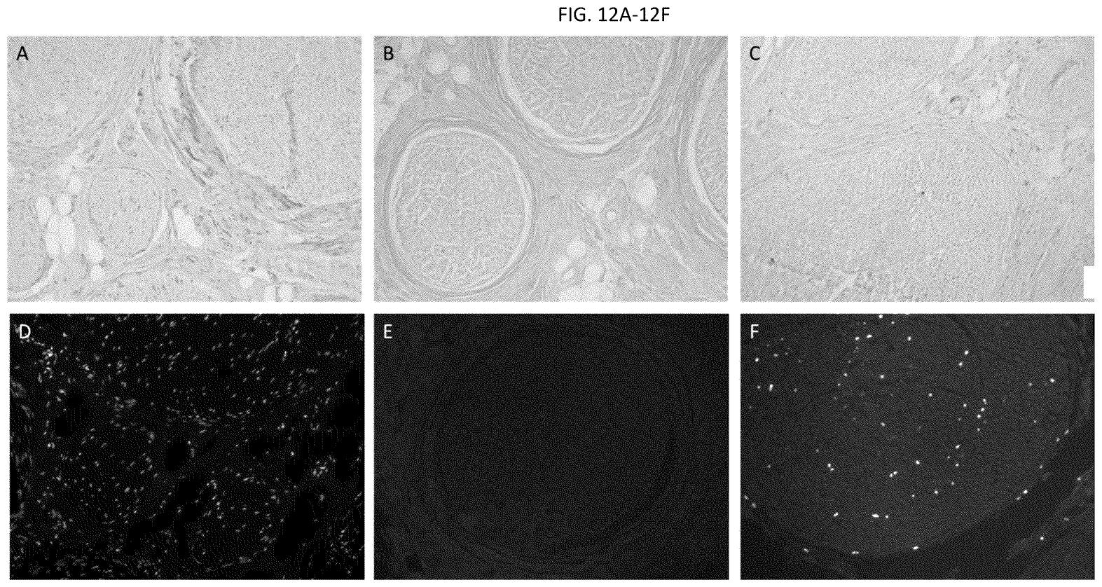

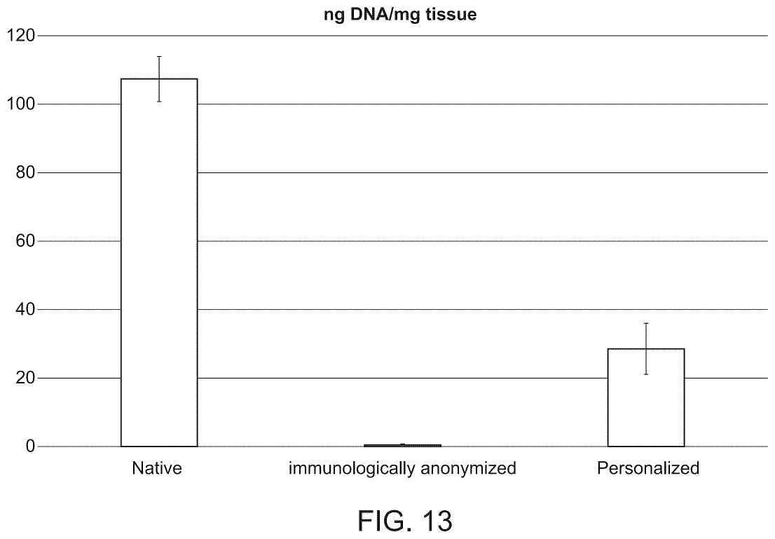

Personalization of anonymized nerve graft scaffolds was analyzed both using histochemical staining (FIG. 12A-12F) of the tissue and DNA quantification (FIG. 13).

FIG. 12A-12F, Evaluation of personalization efficacy. 4,6-diamidino-2-phenylindole (DAPI) and Hematoxylin-Eosin (H&E) staining was performed on cross-sections of 5 μm thickness, following standard protocols. Images were acquired using a fluorescence microscope (Zeiss Axiovert 40 CFL) for DAPI visualization, and in bright field for H&E visualization.

For FIG. 13, DNA was extracted from 10-25 mg wet tissue with the DNeasy Blood & Tissue kit (Qiagen) and quantified with the Qubit dsDNA HS assay kit (Life Technologies), according to the manufacturer's instructions.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A method of preparing an immunologically anonymized nerve segment, comprising:
non-chemically removing cells from a nerve segment; and
enzymatically removing immunogenic remnants from the nerve segment to
provide a immunologically anonymized nerve segment.

2. The method of claim 1, wherein the nerve segment is derived from a human or an animal species.

3. The method of claim 1, further comprising prior to said non-chemically removing, snap-freezing the nerve segment.

4. (canceled)

5. The method of claim 1, wherein said non-chemically removing comprises exposing the nerve segment to a salt solution having a cell disrupting effective concentration of a salt.

6. The method of claim 5, wherein the salt comprises KCl, MgCl₂, NaCl or a combination thereof.

7. (canceled)

8. The method of claim 5, wherein the concentration of the salt in the salt solution is from 0.5 M to 2.0 M.

9. (canceled)

10. The method of claim 1, wherein said non-chemically removing further comprises exposing the nerve segment to a non-osmotic liquid.

11. (canceled)

12. The method of claim 1, wherein the enzymatically removing comprises exposing the nerve segment to an enzyme solution.

13. The method of claim 12, wherein the enzyme solution comprises DNase.

14. (canceled)

15. The method of claim 1, wherein said non-chemically removing and said enzymatically removing are performed sequentially.

16. (canceled)

17. The method of claim 1, further comprising eluting waste products of said non-chemically removing and/or said enzymatically removing from the nerve segment.

18. The method of claim 17, wherein said eluting comprises washing the segment with phosphate-buffered saline.

19. (canceled)

20. The method of claim 1, further comprising sterilizing the immunologically anonymized nerve segment in a peracetic acid solution.

21. (canceled)

22. The method of claim 20, further comprising washing the immunologically anonymized nerve segment in phosphate-buffered saline after sterilization.

23. The method of claim 1, further comprising cryopreserving the immunologically anonymized nerve graft.

24. An immunologically anonymized nerve graft prepared according to claim 1.

25. A method of regenerating a nerve defect, comprising implanting the immunologically anonymized nerve graft prepared by claim 1.

26-27. (Canceled)

28. A method of preparing a personalized nerve graft comprising contacting a nerve graft scaffold with a suspension comprising a whole blood sample from a subject in need of the personalized nerve graft, wherein the whole blood sample is diluted in a physiological solution.

29. (canceled)

30. A method of preparing a personalized nerve graft comprising, contacting a surface of a nerve graft scaffold with an undiluted whole blood sample from a subject in need of the personalized nerve graft.

31-60. (canceled)

61. A method of regenerating a nerve defect, comprising implanting the personalized nerve graft prepared by claim 28 to the subject, whose blood was used for preparing the personalized nerve graft.

62-64. (canceled)
